SEQUENCE LISTING (8 sequences)

SEQ ID NO: 1 - DNA, 2444 bp, artificial sequence
  FLIP insert
gtcgacggatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattctggttgctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaac

SEQ ID NO: 2 - DNA, 103 bp, artificial sequence
  fflux RNAi
aaggtatattgctgttgacagtgagcgagctcccgtgaattggaatcctagtgaagccacagatgtaggattccaattcagcgggagcctgcctactgcctcg

SEQ ID NO: 3 - DNA, 2540 bp, artificial sequence
  pFLIP containing fflux RNAi
gtcgacggatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcaggctcccgctgaattggaatcctacatctgtggcttcactaggattccaattcacgggagctcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaac

SEQ ID NO: 4 - DNA, 7697 bp, modified lentivirus
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcaggctcccgctgaattggaatcctacatctgtggcttcactaggattccaattcacgggagctcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaaacgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 5 - DNA, 103 bp, artificial sequence
  p53 RNAi
aaggtatattgctgttgacagtgagcgcccactacaagtacatgtgtaatgtgaagccacagatgtattacacatgtacttgtagtggatgcctactgcctcg

SEQ ID NO: 6 - DNA, 2530 bp, artificial sequence
  pFLIP containing p53 RNAi
tccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcatccactacaagtacatgtgtaatacatctgtggcttcactattacacatgtacttgtagtgggcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggtt

SEQ ID NO: 7 - DNA, 7698 bp, modified lentivirus
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatccataacttcgtataggataccttatacgaagttatctcaggtaccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccctgtacaagaaacagaaaattgtggcaccagtgaaacagactttgaattttgaccttctcaagttggcgggagacgtcgagtccaaccctgggcccatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcaccctcgggatacccgagcacacgtaccgctcccgcgtcaccctctccaaccagccctatatcaaggtccttaccctagccaacttcaccaccaaggatgagggcgactacttttgtgagcttcgagtctcgggcgcgaatcccatgagctccaataaaagtatcagtgtgtatagagacaaactggtcaagtgtggcggcataagcctgctggttcagaacacatcctggatgctgctgctgctgctttccctctccctcctccaagccctggacttcatttctctgtgatctagaagccataacttcgtatagtacacattatacgaagttatgtttaaacgcattagtcttccaattgaaaaaagtgatttaatttataccattttaattcagctttgtaaaaatgtatcaaagagatagcaaggtattcagttttagtaaacaagataattgctcctaaagtagccccttgaattccgaggcagtaggcatccactacaagtacatgtgtaatacatctgtggcttcactattacacatgtacttgtagtgggcgctcactgtcaacagcaatataccttctcgagccttctgttgggttaacctgaagaagtaatcccagcaagtgtttccaagatgtgcaggcaacgattctgtaaagtactgaagcctcattcaaacatagtatatgtgctgccgaagcgagcacttaacaaggcttgcggccgctacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtataacttcgtataaggtatcctatacgaagttatccattcaggctgtgctagcatcaatggcatggcacaaagcttagccataacttcgtataatgtgtactatacgaagttatcccgggttaaacgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 8 - DNA, 9207 bp, artificial sequence
  pFLIP containing puro2A-GFP
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagacgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtgccaccatggtggagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccaaacagaaaattgtggcaccagtgaaacagactttgaattttgaccttctcaagttggcgggagacgtcgagtccaaccctgggcccggcccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagcggccgcaagccttgttaagtgctcgcttcggcagcacatatactatgtttgaatgaggcttcagtactttacagaatcgttgcctgcacatcttggaaacacttgctgggattacttcttcaggttaacccaacagaaggctcgagcaaccagaattcaaggggctactttaggagcaattatcttgtttactaaaactgaataccttgctatctctttgatacatttttacaaagctgaattaaaatggtataaattaaatcacttttttcaattggaagactaatgcgtttaaacacgcggcgacgcgttcgaccgaataaaacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcggatctcgaccgcgtttgggcggtggctccctgccacgcggctccgaacagaagctgatctccgaagaggatctgacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

(19) United States
(12) Patent Application Publication
(10) Pub. No.: US 2012/0214242 A1
(43) Pub. Date: Aug. 23, 2012

(54) CRE-LOX BASED GENE KNOCKDOWN CONSTRUCTS AND METHODS OF USE THEREOF

(75) Inventors: Patrick Stern, Cambridge, MA (US); Richard Hynes, Winchester, MA (US)

(73) Assignee: MASSACHUSETTS INSTITUTE OF TECHNOLOGY, Cambridge, MA (US)

(21) Appl. No.: 13/349,133

(22) Filed: Jan. 12, 2012

Related U.S. Application Data
(63) Continuation of application No. 11/808,553, filed on Jun. 11, 2007, now abandoned.
(60) Provisional application No. 60/812,608, filed on Jun. 12, 2006.

(51) Int. Cl. C12N 15/85

(52) U.S. Cl. 435/462; 435/320.1

(57) ABSTRACT

The present invention relates to vectors, compositions and methods for conditional, Cre-lox regulated, RNA interference. The vectors allow for spatial and temporal control of miRNA expression in vivo.

### CROSS-REFERENCE TO RELATED APPLICATIONS

This application claims the benefit of U.S. Provisional Application Ser. No. 60/812,608, filed Jun. 12, 2006, which is hereby incorporated herein in its entirety.

### FIELD OF THE INVENTION

The present invention relates to vectors and their use in a cre-lox based method for conditional RNA interference.

### BACKGROUND OF THE INVENTION

RNA interference (RNAi) is an extremely verstatile tool for inhibition of gene expression. RNAi is based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. Short (30 bp) interfering RNA duplexes (siRNA) have been shown to be effective, and do not provoke an immune response, extending the application to mammalian cells. Small hairpin RNAs (shRNAs) transcribed in vivo, are able to trigger degradation of corresponding mRNAs similar to the siRNAs. Micro RNAs (miRNAs) are the endogenous form of shRNAs that carry out the gene silencing function in vivo.

shRNA expression has been accomplished using gene expression vectors, with RNA polymerase III (Pol III) or Polymerase II (Pol II) promoters, with expression occurring in mice injected with the shRNA expression vectors, however, gene inhibition was temporally and spatially restricted. Moreover stable integration of the construct is not readily accomplished or validated in current systems.

### BRIEF DESCRIPTION OF THE DRAWINGS

FIG. 1 depicts one embodiment of the organization and expression of constructs of this invention. A. Shematic representation of an embodiment of the pFLIP inserts of this invention. Two pairs of mutated loxP sites and their orientation are depicted, as well as the positioning of the positioning of the selectable marker sequences and miRNA sequence, with respect thereto. Schematic shows elements prior to and following Cre-mediated recombination.

FIG. 2 shows the results of a FACS analysis of pFLIP expression in “uninduced” and “induced” states in Lewis Lung carcinoma cell lines (LL2) infected with an MSCV retrovirus expressing pFLIP. The panels show FACS analysis for expressed markers.

FIG. 3 demonstrates expression and knockdown ability of an embodiment of the retroviral pFLIP vectors of this invention in the presence of Cre. Primary mouse embryo fibroblasts (MEFs) were infected with MSCV retrovirus expressing pFLIP comprising an RNAi against the tumor suppressor p53. Cells were selected with puromycin and infected with retrovirus expressing Cre recombinase. Cells were treated for 5 hours with doxorubicin, which causes DNA damage and induces p53 expression.

### DETAILED DESCRIPTION OF THE INVENTION

The present invention provides, in one embodiment, constructs and methods for conditionally reducing expression of a coding sequence in a cell or animal, comprising contacting the cell with a vector comprising a first selectable marker in sense orientation, and a second selectable marker fused in frame to an miRNA sequence, in antisense orientation, whereby the marker sequences are flanked by two pairs of loxP sites, which sites are initially inverted in orientation, in cells capable of expressing a Cre recombinase.

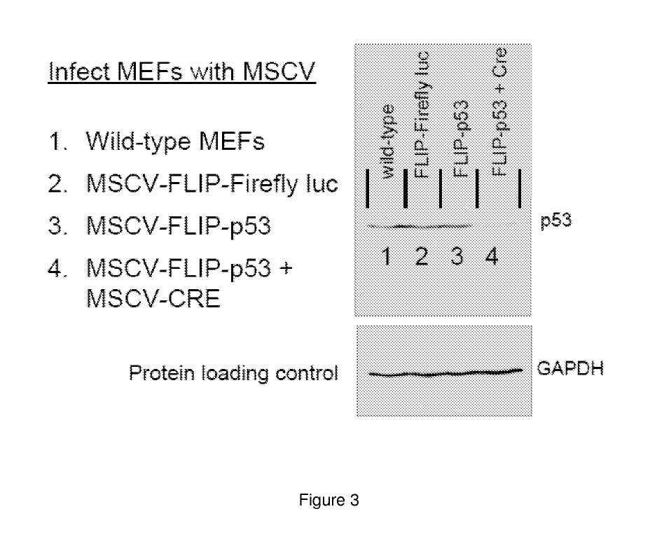

Conditionally reduced expression of a coding sequence was demonstrated herein, with the use of a retroviral vector pFLIP, which comprises, in some embodiments, a first pair of loxP sequences, inverted in orientation, with respect to each other, a first nucleic acid encoding a first selectable marker in sense orientation, wherein said nucleic acid is positioned between said first pair of loxP sequences, a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid, a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences. Cre expression in MEF cells infected with pFLIP expressed the shRNA, and demonstrated reduced p53 protein levels (FIG. 3).

In one embodiment, this invention provides a method of conditionally reducing expression of a coding sequence in a target cell, said method comprising contacting said target cell with a vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences;
   wherein said target cell is capable of expressing a Cre recombinase and whereby, following Cre-mediated recombination, said miRNA agent is expressed and reduces expression of said coding sequence, thereby conditionally reducing expression of a coding sequence in a target cell.

In one embodiment, the term “vector” refers to a nucleic acid molecule capable of transporting another nucleic acid to which it has been linked. In one embodiment, the vector is a genomic integrated vector, or “integrated vector”, which can become integrated into the chromsomal DNA of the host cell. In another embodiment, the vector is an episomal vector, i.e., a nucleic acid capable of extra-chromosomal replication in an appropriate host, such as, for example a eukaryotic host cell. The vector according to this aspect of the present invention may be, in other embodiments, a plasmid, a bacmid, a phagemid, a cosmid, a phage, a virus or an artificial chromosome.

A nucleic acid of the present invention will generally contain phosphodiester bonds in one embodiment, or in another embodiment, nucleic acid analogs are included, that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′O-methyl and 5′ modified substituents, or to increase the stability and half-life of such molecules in physiological environments.

The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996).

The vectors of this invention comprise, inter alia, an miRNA agent specific for a coding sequence.

The term “miRNA agent” refers, in one embodiment, to an agent that modulates expression of a target gene by an RNA interference mechanism. Micro-RNAs are a very large group of small RNAs produced naturally in organisms, which in one embodiment, regulates the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ^{˜}70 nt precursor, which is post-transcriptionally processed into a mature ^{˜}21 nt form. Both let-7 and lin-4 are transcribed as hairpin RNA precursors, which are processed to their mature forms by Dicer enzyme.

In one embodiment the miRNA agent comprises double-stranded RNA, which can form a hairpin structure. The miRNA agents employed, in another embodiment, are small ribonucleic acid molecules, or oligoribonucleotides, that are present in duplex structures, such as, in one embodiment, two distinct oligoribonucleotides hybridized to each other, or in another embodiment, a single ribooligonucleotide that assumes a hairpin structure to produce a duplex structure.

In one embodiment, miRNA agent does not exceed about 100 nt in length, and typically does not exceed about 75 nt length, where the length in certain embodiments is less than about 70 nt. In one embodiment, the miRNA agent of this invention has a length about 15 to 40 bp, or in another embodiment, about 20 and 29 bps, or in another embodiment, 25 and 35 bps, or in another embodiment, about 20 and 35 bps, or in another embodiment, about 20 and 40 bps, or in another embodiment, 21 bp, or in another embodiment, 22 bp.

In one embodiment, the nucleic acids/oligonucleotides comprising the miRNA agent may be synthesized on an Applied Bio Systems oligonucleotide synthesizer according to specifications provided by the manufacturer. In another embodiment, the nucleic acids/oligonucleotides or modified oligonucleotides may be synthesized by any number of means as is generally known in the art, and as is described hereinbelow.

In one embodiment, the miRNA agent encodes an interfering ribonucleic acid. In one embodiment, the miRNA agent is a transcriptional template of the interfering ribonucleic acid. According to this aspect of the invention, and in one embodiment, the transcriptional template is typically a DNA that encodes the interfering ribonucleic acid. The DNA may be present in a vector, such as, and in one embodiment, a plasmid vector, or in another embodiment, a viral vector, or any other vector, as will be known to one skilled int the art.

In one embodiment, the term “coding sequence” refers to a nucleic acid sequence that “encodes” a particular polypeptide or peptide. In one embodiment, the coding sequence is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

In one embodiment the term “coding sequence”, includes DNA sequences that encode a polypeptide, as well as DNA sequences that are transcribed into inhibitory antisense molecules.

In one embodiment, the term “reducing expression”, as it refers to vectors and their use according to the methods of this invention, refers to a diminishment in the level of expression of a gene when compared to the level in the absence of the miRNA agent.

In one embodiment, reduced expression may be affected at the transcriptional or translational level, or a combination thereof.

According to this aspect of the invention, reduced expression using the vectors, and/or according to the methods of this invention, is specific. In one embodiment, the reduction in expression is via an ability to inhibit a target gene without manifest effects on other genes of the cell. The consequences of inhibition can be confirmed, in other embodiments, by examination of the outward properties of the cell or organism or by biochemical techniques such as RNA solution hybridization, nuclease protection, Northern hybridization, gene expression monitoring with a microarray, antibody binding, enzyme linked immunosorbent assay (ELISA), Western blotting, radioimmunoassay (RIA), other immunoassays, and fluorescence activated cell analysis (FACS).

In one embodiment, the miRNA agent is an shRNA, which specifically inactivates p53, as exemplified hereinbelow.

In one embodiment, the vectors and methods of utilizing the same for reducing expression of a target gene may result in inhibition of target gene expression of greater than 10%, 33%, 50%, 75%, 80%, 85%, 90%, 95% or 99% as compared to a cell not subjected to the vectors and methods of utilizing the same for reducing expression. In another embodiment, lower doses of administered miRNA agent, and longer times following administration may result in inhibition in a smaller fraction of cells (e.g., at least 10%, 20%, 50%, 75%, 90%, or 95% of targeted cells).

In one embodiment, this invention provides for a method of conditionally reduced expression of a coding sequence in a target cell. In one embodiment, the term “conditionally reduced expression” refers to the flexibility inherent in the methods/vectors of this invention, which enable regulation of reducing expression of a coding sequence in a target cell. In one embodiment, reducing expression via the vectors/methods of this invention is controlled over time, or in a cell or tissue-specific manner, such that production of the miRNA agent is not constant.

Expression of the miRNA agent within a target cell, in one embodiment of this invention, takes advantage of a lox/cre system. In one embodiment, miRNA agent expression is dependent upon the presence of a Cre recombinase. In one embodiment, abrogation of miRNA expression is dependent upon the presence of a Cre recombinase.

In one embodiment, the cre recombinase, is derived from a P1 bacteriophage (Abremski and Hoess, J. Biol. Chem. 259(3):1509-1514 (1984)) which acts on a specific 34 base pair DNA sequence known as “loxP” (locus of crossover), which is, in turn, comprised of two 13 base pair inverted repeats (serving as the recombinase binding sites) flanking an 8 base pair core sequence (Current Opinion in Biotechnology 5:521-527 (1994). Cre catalyzes the rearrangement of DNA sequences that contain loxP sites. Recombination between two loxP sites (catalyzed by the cre protein) causes, in certain cases, the loss of sequences flanked by these sites [for a review see N. Kilby et al, Trends Genet., 9:413-421 (1993)].

In some embodiments, this invention utilizes two sets of loxP sites, whose sequences differ. In one embodiment, one pair of the loxP sites may be WT, while the other may be mutated, or in another embodiment, both are mutated. In one embodiment, the loxP sets are oriented initially with inverted orientation, such that regions of the vector undergo inversion, following exposure to a Cre-recombinase. Following such inversion, one of the pairs of loxP sites are co-aligned, thus in the presence of a Cre-recombinase, excision can occur.

In one embodiment, the two pairs of loxP sites are chosen so as to minimize recombination therebetween, as exemplified herein.

Cre works in simple buffers, such as, in one embodiment, with magnesium or, in another embodiment, spermidine as a cofactor, as is well known in the art. The DNA substrates acted on by cre may be, in one embodiment, in linear, or, in another embodiment, in a supercoiled configuration.

In one embodiment, the cre sequence is as that described in N. Sternberg et al, J. Mol. Biol., 187:197-212 (1986). In another embodiment, the cre recombinase may be obtained from commercial sources (for example from Novagen, Catalog No. 69247-1).

In one embodiment, cre recombinase will be expressed in a target cell of this invention. In another embodiment, the target cell will be engineered to express cre by any means as will be known to one skilled in the art.

In one embodiment, the terms “homology”, “homologue” or “homologous”, refer to a, which exhibits, in one embodiment at least 70% correspondence with the indicated sequence. In another embodiment, the sequence exhibits at least 72% correspondence with the indicated sequence. In another embodiment, the sequence exhibits at least 75% correspondence with the indicated sequence. In another embodiment, the sequence exhibits at least 80% correspondence with the indicated sequence. In another embodiment, the sequence exhibits at least 82% correspondence with the indicated sequence. In another embodiment, the sequence exhibits at least 85% correspondence with the indicated sequence. In another embodiment, the sequence exhibits at least 87% correspondence with the indicated sequence. In another embodiment, the sequence exhibits at least 90% correspondence with the indicated sequence. In another embodiment, the sequence exhibits at least 92% correspondence with the indicated sequence. In another embodiment, the sequence exhibits at least 95% or more correspondence with the indicated sequence. In another embodiment, the sequence exhibits at least 97% correspondence with the indicated sequence. In another embodiment, the sequence exhibits at least 99% correspondence with the indicated sequence. In another embodiment, the sequence exhibits 95%-100% correspondence with the indicated sequence. Similarly, as used herein, the reference to a correspondence to a particular sequence includes both direct correspondence, as well as homology to that sequence as herein defined.

Homology, as used herein, may refer to sequence identity, or may refer to structural identity, or functional identity. By using the term “homology” and other like forms, it is to be understood that any molecule, that functions similarly, and/or contains sequence identity, and/or is conserved structurally so that it approximates the reference sequence, is to be considered as part of this invention.

Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, Nucleic Acid Hybridization, Hames and Higgins, Eds. (1985); Molecular Cloning, Sambrook and Russell, eds. (2001), and Current Protocols in Molecular Biology, Ausubel et al. eds, 1989). For example, methods of hybridization may be, in one embodiment, carried out under moderate to stringent conditions, to the complement of a DNA encoding a native peptide or protein of interest. Hybridization conditions may be, for example, overnight incubation at 42° C. in a solution comprising: 10-20% formamide, 5×SSC (150 millimolar (mM) NaCl, 15 mM trisodium citrate), 50 mM sodium phosphate (pH 7.6), 5×Denhardt's solution, 10% dextran sulfate, and 20 micrograms (μg)/milliliter (ml) denatured, sheared salmon sperm DNA. Each method represents a separate embodiment of the present invention.

In another embodiment, mutated loxP sites, may be employed in the vectors and/or methods of this invention.

In one embodiment, the constructs of this invention will comprise a promoter, operatively linked to the first nucleic acid sequence encoding a selection marker. In one embodiment, the term “promoter” refers to a nucleic acid sequence, which regulates expression of a nucleic acid, operably linked thereto. Such promoters are known to be cis-acting sequence elements required for transcription as they serve to bind DNA dependent RNA polymerase, which transcribes sequences present downstream thereof.

The term “operably linked”, in one embodiment, refers to a relationship permitting the sequences to function in their intended manner. A vector comprising a regulatory sequence “operably linked” to a coding sequence is ligated in such a way that expression of the nucleic acid sequence is achieved under conditions compatible with the control sequences.

In one embodiment, the promoter will be an RNA polymerase III promoter.

In one embodiment, a promoter, including an engineered promoter used in the vectors and methods of this invention, may be one known to confer cell-type specific expression of a sequence operatively linked to thereto. For example, and in one embodiment, a promoter specific for myoblast gene expression can be operatively linked to an miRNA for a coding sequence of interest, a reporter gene, or a coding sequence of interest, to confer muscle-specific expression thereof. Muscle-specific regulatory elements which are known in the art include upstream regions from the dystrophin gene (Klamut et al., (1989) Mol. Cell. Bio1.9:2396), the creatine kinase gene (Buskin and Hauschka, (1989) Mol. Cell. Biol. 9:2627) and the troponin gene (Mar and Ordahl, (1988) Proc. Natl. Acad. Sci. USA. 85:6404).

In another embodiment, promoters used in the vectors and methods of this invention, specific for other cell types known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters) may be used, and represent an embodiment of this invention. In another embodiment, a promoter or regulatory element, which can direct constitutive expression of a sequence operatively linked thereto, in a variety of different cell types, such as a viral regulatory element, may be used. Examples of viral promoters commonly used to drive gene expression include those derived from polyoma virus, Adenovirus 2, cytomegalovirus and Simian Virus 40, and retroviral LTRs.

In another embodiment, a regulatory element, which provides inducible expression of a gene linked thereto, may be used. The use of an inducible promoter may allow, in another embodiment, for an additional means of modulating the product of the coding sequence in the cell. Examples of potentially useful inducible regulatory systems for use in eukaryotic cells include hormone-regulated elements (e.g., see Mader, S, and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89: 1014-10153). Additional tissue-specific or inducible regulatory systems may be developed for use in accordance with the invention.

In one embodiment, the term “capable of expressing a Cre recombinase” refers to a cell that endogenously expresses the Cre recombinase, or in another embodiment, is engineered to express a Cre recombinase.

In one embodiment, the cell is in a culture system, or in another embodiment, in a body of a subject, or in another embodiment, is ex-vivo cultured, and following transfection or transduction with a vector of this invention, is reintroduced to the subject from which the cell was taken. In one embodiment, the cell is a stem or progenitor cell. In another embodiment, the cell is a mature, differentiated cell. In one embodiment, the cell is a human cell in origin, or in another embodiment, the cell is murine in origin.

In one embodiment, the terms “Cells,” “host cells” or “target cells” are used interchangeably, and refer, in one embodiment, not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

In another embodiment, the cell is a diseased cell. In one embodiment, the cell is infected, or in another embodiment, the cell is transformed or neoplastic. In another embodiment, the cell is obtained from a subject with a disease whose etiology is associated with a genetic mutation. In another embodiment, the cell is obtained from a subject with a disease, where an inappropriate immune or inflammatory response has been initiated.

In one embodiment, the target cell of any method of the present invention may be a cancer cell or neoplastic cell. “Neoplastic cell” refers, in one embodiment, to a cell whose normal growth control mechanisms are disrupted (typically by accumulated genetic mutations), thereby providing potential for uncontrolled proliferation. Thus, “neoplastic cell” can include, in one embodiment, both dividing and non-dividing cells. In one embodiment, neoplastic cells may include cells of tumors, neoplasms, carcinomas, sarcomas, leukemias, lymphomas, and others. In another embodiment, “neoplastic cells” may include central nervous system tumors, such as, for example brain tumors. These may include, in other embodiments, glioblastomas, astrocytomas, oligodendrogliomas, meningiomas, neurofibromas, ependymomas, schwannomas or neurofibrosarcomas. In another embodiment, “neoplastic cells” can include either benign or malignant neoplastic cells. In another embodiment, “neoplastic cells” can include any other type of cancer known in the art.

In one embodiment, the target cell may be an infected cell. In another embodiment, the target cell may be a pathogenic cell. In another embodiment, the target cell may mediate autoimmunity or another disease state. In another embodiment, the target cell may comprise a mutated cellular gene necessary for a physiological function. In one embodiment, the mutated product results in disease in the subject. According to this aspect of the invention, the vectors/methods of this invention may be employed to silence a defective gene, and may further be followed by delivery of a wild-type copy of the desired gene.

It is to be understood that any cell comprising a vector of this invention, or utilized for the methods of this invention, is to be considered as part of this invention, and represents an embodiment thereof.

According to this aspect of the invention, and in one embodiment, following Cre-mediated recombination in the target cell, the miRNA agent is expressed and reduces expression of the coding sequence, thereby conditionally reducing expression of a coding sequence in the target cell.

In another embodiment, the vector is a retroviral vector. In one embodiment, the retroviral vector of this invention may correspond to one as exemplified herein.

A retroviral or retrovirus vector, as used herein, is a vector, which comprises at least one component part derivable from a retrovirus. In one embodiment, the component part is involved in the biological mechanisms by which the vector infects cells, expresses genes or is replicated. The term “derivable”, in one embodiment, refers to the fact that the sequence need not necessarily be obtained from a retrovirus but instead could be derived therefrom. By way of example, the sequence may be prepared synthetically or by use of recombinant DNA techniques.

The retroviral vectors of this invention may be derived from any member of the family of retroviridae.

In one embodiment, the retroviral vectors of this invention comprise sufficient retroviral genetic information to allow packaging of an RNA genome, in the presence of packaging components, into a viral particle capable of infecting a target cell. In one embodiment, infection of the target cell includes reverse transcription and integration into the target cell genome. The retroviral vectors of this invention may carry, in one embodiment, non-viral coding sequences which are to be delivered by the vector to the target cell. In one embodiment, the retroviral vectors of this invention are incapable of independent replication to produce infectious retroviral particles within the final target cell. In one embodiment, the retroviral vectors of this invention will lack a functional gag-pol and/or env gene and/or other genes essential for replication.

In one embodiment, the vectors and methods of this invention may employ the use of enhancer sequences. In one embodiment, the term “enhancer” refers to a DNA sequence, which binds to other protein components of the transcription initiation complex and may thus facilitate the initiation of transcription directed by its associated promoter.

In another embodiment, the vectors and their use according to the present invention include at least two selectable markers, which may serve to indicate inversion and excision mediated by a Cre-recombinase, as described herein. In one embodiment, the selectable marker comprises an antibiotic resistance cassette, by means well known to one skilled in the art. In one embodiment, the resistance cassette is for conferring resistance to ampicillin, bleomycin, chloramphenicol, gentamycin, hygromycin, kanamycin, lincomycin, methotrexate, phosphinothricin, puromycin, or tetracycline, or derivatives thereof.

In another embodiment, the selectable marker may comprise nucleic acid sequences encoding for a reporter protein, such as, for example, green fluorescent protein (GFP), DS-Red (red fluorescent protein), acetohydroxyacid synthase (AHAS), beta glucoronidase (GUS), secreted alkaline phosphatase (SEAP), beta-galactosidase, chloramphenicol acetyltransferase (CAT), horseradish peroxidase (HRP), luciferase, nopaline synthase (NOS), octopine synthase (OCS), or derivatives thereof, or any number of other reporter proteins known to one skilled in the art.

In another embodiment, the vector may further include an origin of replication, and may be a shuttle vector, which can propagate both in bacteria, such as, for example, E. coli (wherein the vector comprises an appropriate selectable marker and origin of replication) and be compatible for propagation in vertebrate cells, or integration in the genome of an organism of choice.

The nucleic acids may be introduced into tissues or host cells by any number of routes, including viral infection, microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. Expression vectors may be used to introduce the nucleic acids into a cell.

In one embodiment, the vectors of this invention may be fed directly to, injected into, the host organism containing the target gene. The vectors of this invention may be directly introduced into the cell (i.e., intracellularly); or introduced extracellularly into a cavity, interstitial space, into the circulation of an organism, introduced orally, etc. Methods for oral introduction include direct mixing of the vector with food of the organism. Physical methods of introducing the vectors include injection directly into the cell or extracellular injection into the organism of a solution comprising the vector. The vectors may be introduced in an amount, which allows delivery of at least one copy per cell. Higher doses (e.g., at least 5, 10, 100, 500 or 1000 copies per cell) of the vectors may yield more effective inhibition; lower doses may also be useful for specific applications.

In other embodiments, a hydrodynamic administration protocol is employed, and may be as described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349, each of which represents an embodiment of this invention.

In other embodiments, delivery protocols of interest may include, but are not limited to: those described in U.S. Pat. Nos. 5,985,847, or 5,922,687, WO/11092;. Acsadi et al., New Biol. (1991) 3:71-81; Hickman et al., Hum. Gen. Ther. (1994) 5:1477-1483; or Wolff et al., Science (1990) 247: 1465-1468, and others, as will be appreciated by one skilled in the art.

The methods of this invention comprise the step of contacting a target cell with a vector of this invention. In one embodiment, the terms “contacting”, “contact” or “contacted” indicate, direct or, in another embodiment, indirect exposure of the cell to a vector, compound or composition comprising the vectors of this invention. It is envisaged that, in another embodiment, indirect supply to the cell may be via provision in a culture medium that surrounds the cell, or via parenteral administration in a body of a subject, whereby the vector ultimately contacts a cell via peripheral circulation (for further detail see, for example, Methods in Enzymology Vol. 1-317, Rubin and Dennis, eds, (1955-2003) and Current Protocols in Molecular Biology, Ausubel, et al, eds (1998), Molecular Cloning: A Laboratory Manual, Sambrook and Russell, eds., (2001), or other standard laboratory manuals). It is to be understood that any direct means or indirect means of intracellular access of a vector, or composition comprising the same of this invention represents an embodiment thereof.

In one embodiment, the target cell is contacted with a vector/composition comprising the same, of this invention, in vivo, in vitro or ex-vivo. In one embodiment, cells may be procured from a subject, contacted with a vector of this invention, and reintroduced into the subject. In one embodiment, the cell is a stem or progenitor cell, and reintroduction into the subject may be followed, in another embodiment, by stimulation of differentiation of the contacted cell, in vivo.

In another embodiment, Cre recombinase is expressed at specific times during development.

In another embodiment, this invention provides for the generation of a non-human animal with reduced expression of a coding sequence, wherein the reduced expression is produced according to the methods, and/or utilizing the vectors of this invention.

Transgenic mice, may, in one embodiment, be derived using the vectors/methods of this invention, according to Hogan, et al., “Manipulating the Mouse Embryo: A Laboratory Manual”, Cold Spring Harbor Laboratory (1988) which is incorporated herein by reference. Embryonic stem cells may, in another embodiment, be manipulated according to published procedures (Teratocarcinomas and embryonic stem cells: a practical approach, E. J. Robertson, ed., IRL Press, Washington, D.C., 1987; Zjilstra et al., Nature 342:435-438 (1989); and Schwartzberg et al., Science 246:799-803 (1989), each of which is incorporated herein by reference). Zygotes may be manipulated, in another embodiment, according to known procedures; for example see U.S. Pat. No. 4,873,191, Brinster et al., PNAS 86:7007 (1989); Susulic et al., J. Biol. Chem. 49:29483 (1995), and Cavard et al., Nucleic Acids Res. 16:2099 (1988), hereby incorporated by reference. Tetraploid blastocyst complementation may also be utilized to achieve non-human animals, which express the vectors of this invention, according to methods as exemplified herein, or, as are well known in the art.

In one embodiment, this invention provides a method of producing an animal genetically inactivated for a coding sequence, the method comprising contacting an embryonic stem cell with a vector of this invention which may be used for gene silencing, injecting the contacted embryonic stem cell to a blastocyst of an animal and obtaining an animal expressing the vector, whereby, following Cre-mediated recombination in the animal, the miRNA agent is expressed and reduces expression of the coding sequence, thereby being a method of producing an animal genetically inactivated for a coding sequence.

In another embodiment, the method of conditionally reducing expression of a coding sequence, as described and exemplified herein, may be therapeutic. In one embodiment, the term “therapeutic” refers to the fact that when in contact with a cell in a subject in need, provides a beneficial effect.

In one embodiment, the compositions/vectors and methods of conditionally reducing expression of a coding sequence of this invention prevent inappropriate expression of an encoded protein in a subject. Some examples include endogenous proteins which are mutated, and produces a non-functional protein, or an over-expressed protein, which in another embodiment, may be non-functional, or in another embodiment, pathogenic.

In one embodiment, the encoded protein may include cytokines, such as interferons or interleukins, or their receptors. According to this aspect of the invention, and in one embodiment, inappropriate expression patterns of cytokines may be altered to produce a beneficial effect, such as for example, a biasing of the immune response toward a Th1 type expression pattern, or a Th2 pattern in infection, or in autoimmune disease, wherein altered expression patterns may prove beneficial to the host. In these cases, and in one embodiment, conditionally reducing expression of the inappropriate or non-protective cytokine/receptor may be followed by delivery of an appropriate cytokine, or a vector/nucleic acid for expressing the same.

In another embodiment, the encoded protein may include an enzyme, such as one involved in glycogen storage or breakdown. In another embodiment, the encoded protein may include a transporter, such as an ion transporter, for example CFTR, or a glucose transporter, or other transporters whose inappropriate expression results in a variety of diseases. As described hereinabove, and in another embodiment, conditionally reducing expression of the encoded proteins, according to this aspect of the invention, may be followed by delivery of a wild-type protein, or a plasmid encoding same, or a mutated protein, which results in a therapeutic effect in the subject.

In another embodiment, the encoded protein may include a receptor, such as one involved in signal transduction within a cell. Some examples include as above, cytokine receptors, leptin receptors, transferring receptors, etc., or any receptor wherein altered expression results in inappropriate or inadequate signal transduction in a cell.

It is to be understood that any encoded protein, wherein conditionally reducing expression of the product is therapeutic to a subject is to be considered as part of this invention, and methods/vectors to provide wild-type or otherwise therapeutic versions of the encoded protein to the subject, following conditional reduction of expression of the mutated version, is to be considered as part of this invention, and embodiments thereof.

In another embodiment, the vectors/methods of this invention may be utilized to conditionally reduce expression of an oncogene, whose expression promotes cancer-related events. In one embodiment, the conditionally reduced expression of oncogenes comprising ABLI, BCLI, BCL2, BCL6, CBFA2, CBL, CSFIR, ERBA, ERBB, EBRB2, ETSI, ETS1, ETV6, FOR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, YES, or combinations thereof, may be effected via the vectors/compositions/methods of this invention. In another embodiment, vectors/methods of this invention may be utilized to conditionally reduce expression of a Prostate Tumor Inducing Gene, which may comprise in one embodiment, PTI-1, PTI-2, PTI-3 or combinations thereof.

In one embodiment, the vectors/methods of this invention may be utilized to conditionally reduce expression of genes whose products promote angiogenesis, such as, for example, and in one embodiment, VEGF, VEGF receptor, erythropoietin, or combinations thereof. In another embodiment, the coding sequence for which conditional reducing expression is desired may comprise a matrix metalloproteinase, wherein reduction of expression prevents, in one embodiment, metastasis of cancerous cells, or, in another embodiment, tissue necrosis in infectious or inflammatory diseases.

In another embodiment, the vectors/compositions/methods of this invention may be utilized to conditionally reduce expression of a mutated rhodopsin gene. Autosomal dominant retinitis pigmentosa (ADRP) is characterized by the substitution of histidine for proline at codon 23 (P23H) in their rhodopsin gene, resulting in photoreceptor cell death from the synthesis of the abnormal gene product. In one embodiment, P23H mutant mRNAs may be targeted for conditional reduction of expression.

In another embodiment, the vectors/compositions/methods of this invention may be utilized to reverse effects of high glucose on progression of diabetic retinopathy. High glucose environments can result in chronically increased nitric oxide (NO) activity, which leads to endothelial cell dysfunction and impaired blood retinal barrier integrity characteristic of diabetic retinopathy.

In one embodiment, NOS synthesis may be conditionally reduced, in a tissue specific manner, in another embodiment, via the use of miRNAs targeted against VEGF, iNOS, or eNOS using the vectors/compositions and methods, as described hereinabove. In another embodiment, glucose transporters may be similarly targeted for therapeutic purposes in diabetic retinopathy.

In another embodiment, the vectors/compositions and methods for reducing expression of a coding sequence may be applied in a subject with a disease, where the disease may comprise, but is not limited to: muscular dystrophy, cancer, cardiovascular disease, hypertension, infection, renal disease, neurodegenerative disease, such as alzheimer's disease, parkinson's disease, huntington's chorea, Creurtfeld-Jacob disease, autoimmune disease, such as lupus, rheumatoid arthritis, endocarditis, Graves' disease or ALD, respiratory disease such as asthma or cystic fibrosis, bone disease, such as osteoporosis, joint disease, liver disease, disease of the skin, such as psoriasis or eczema, ophthalmic disease, otolaryngeal disease, other neurological disease such as Turret syndrome, schizophrenia, depression, autism, or stoke, or metabolic disease such as a glycogen storage disease or diabetes. It is to be understood that any disease whereby reduced expression of a particular protein, which can be accomplished via the use of the vectors or cells or compositions, or via the methods of this invention, is to be considered as part of this invention.

In another embodiment, this invention provides a method of conditionally expressing a coding sequence in a target cell, the method comprising contacting the target cell with a vector comprising:
a. i. a first pair of loxP sequences, inverted in orientation, with respect to each other;
b. a first nucleic acid encoding a first selectable marker in sense orientation, fused in frame to an miRNA sequence of interest, wherein said nucleic acid is positioned between said first pair of loxP sequences;
c. a second nucleic acid encoding a second selectable marker, in antisense orientation, said second nucleic acid is positioned between said first pair of loxP sequences, and said second nucleic acid is 3′ with regard to said first nucleic acid;
d. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.
wherein said cell expresses said miRNA agent, thereby reducing expression of said coding sequence and whereby, following Cre-mediated recombination in said target cell, said miRNA agent is no longer expressed, thereby being a method of conditionally expressing a coding sequence in a target cell.

It is to be understood that any embodiment, or permutation thereof, described for a method/vector/composition hereinabove, in reference to conditionally reducing expression of a coding sequence, may be applied to that of the vectors/compositions or methods of conditionally expressing a coding sequence, and represent embodiments of this invention.

According to this aspect of the invention and in another embodiment, this invention provides a method of producing an animal genetically reactivated for a coding sequence, the method comprising contacting an embryonic stem cell with a vector for conditionally expressing a coding sequence, injecting the embryonic stem cell to a blastocyst of the animal, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In another embodiment, this invention provides a method of producing an animal genetically reactivated for a coding sequence, the method comprising contacting a single cell embryo of the animal a vector for conditionally expressing a coding sequence, and obtaining an animal expressing the vector, whereby, following Cre-mediated recombination in the animal, the miRNA agent is no longer expressed and the coding sequence is expressed, thereby being a method of producing an animal genetically reactivated for a coding sequence.

In one embodiment, conditional expression of the coding sequence is accomplished at a specific developmental stage. Such expression may be accomplished, in one embodiment, via delivery of a cre recombinase to a desired cell at a specific developmental stage, or in another embodiment, the cre recombinase is present in the cell, under the control of an inducible promoter, and cre expression is induced at a specific developmental stage. In another embodiment, conditional expression of the coding sequence is accomplished in specific tissues or cells, via similar methodology, or in another embodiment, via targeted delivery of a cre recombinase to a particular cell, such as, for example via delivery in a pseudotyped viral vector, which specifically infects a desired cell type.

In another embodiment, the coding sequence for which conditional expression is desired may comprise insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the α family, transforming growth factors of the β family, gastrins, secretins, cholecystokinins, somatostatins, serotonins, substance P and transcription factors and enzymes (e.g., ACC synthases and oxidases, ACP desaturases and hydroxylases, ADP-glucose pyrophorylases, ATPases, alcohol dehydrogenases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase), or combinations thereof.

In another embodiment, the coding sequence for which conditional expression is desired may comprise a tumor suppressor gene, such as, for example, APC, BRCA 1, BRCA2, MADH4, MCC, NF 1, NF2, RB 1, TP53, WTI, or combinations thereof. Conditional expression of these genes, in turn, may in one embodiment, suppress, or in another embodiment, diminish severity, or in another embodiment, prevent metastasis of a cancer.

In another embodiment, the coding sequence for which conditional expression is desired may comprise an immunomodulating protein, such as, for example, cytokines, chemokines, complement components, immune system accessory and adhesion molecules or their receptors, such as, for example, GM-CSF, IL-2, IL-12, OX40, OX40L (gp34), lymphotactin, CD40, and CD40L, interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, complement components and their receptors, or an accessory molecule such as B7.1, B7.2, TRAP, ICAM-1, 2 or 3, cytokine receptors, OX40, OX40-ligand (gp34), or combinations thereof.

In another embodiment, the coding sequence for which conditional expression is desired may comprise a protein, which suppresses angiogenesis. Such a scenario is desirable in a number of disease states, including cancer, hemangiomas, glaucoma, and other diseases, as will be well known to one skilled in the art. In one embodiment, suppression of angiogenesis is accomplished via conditionally expressing an endostatin.

In another embodiment, the methods/vectors/compositions of this invention do not exhibit the limitation of causing constitutive gene silencing or gene expression, in all tissues. According to this aspect of the invention, the methods of this allow for regulated expression of miRNA and thereby regulated expression of a desired coding sequence.

In another embodiment, this invention provides for kits for conditional reduction of expression, or conditional expression of a coding sequence, comprising one or more containers filled with one or more of the ingredients of the aforementioned vectors, or compositions of the invention.

The vectors of the invention may be employed, in another embodiment, in combination with a non-sterile or sterile carrier or carriers for administration to cells, tissues or organisms, such as a pharmaceutical carrier suitable for administration to an individual. Such compositions comprise, for instance, a media additive or a therapeutically effective amount of a recombinant virus of the invention and a pharmaceutically acceptable carrier or excipient. Such carriers may include, but are not limited to, saline, buffered saline, dextrose, water, glycerol, and combinations thereof. The formulation should suit the mode of administration.

The vectors or compositions of the invention may be employed alone or in conjunction with other compounds, such as additional therapeutic compounds.

The pharmaceutical compositions may be administered in any effective, convenient manner including, for instance, administration by intravascular (i.v.), intramuscular (i.m.), intranasal (i.n.), subcutaneous (s.c.), oral, rectal, intravaginal delivery, or by any means in which the recombinant virus/composition can be delivered to tissue (e.g., needle or catheter). Alternatively, topical administration may be desired for insertion into epithelial cells. Another method of administration is via aspiration or aerosol formulation.

For administration to mammals, and particularly humans, it is expected that the physician will determine the actual dosage and duration of treatment, which will be most suitable for an individual and can vary with the age, weight and response of the particular individual.

The following examples are presented in order to more fully illustrate some embodiments of the invention. They should, in no way be construed, however, as limiting the scope of the invention.

### EXAMPLES

### Materials and Methods

### Generation of Constructs

To generate pFLIP, the pMIG vector (Grez et al., Proc. Natl. Acad. Sci. USA 87:9202-9206, 1990), was modified, as follows: the vector was digested with BglII and SalI, the FLIP insert replaced ires-GFP, followed by insert fill in and ligation.

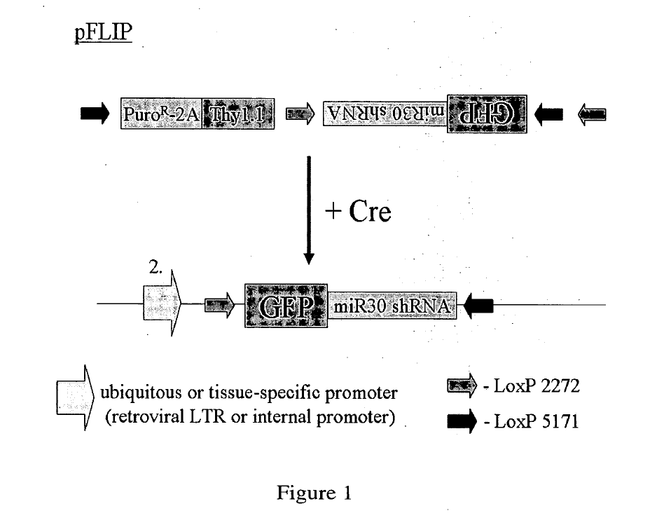

The FLIP insert comprises loxP 5171 and loxP 2272 genes, a modified puromycin resistance cassette incorporating the foot-and-mouth-disease virus (FMDV) 2A encoding sequence at its C-terminus, fused in frame to a gene encoding the Thy1.1 surface marker (See Schnutgen F, et. al., Nat. Biotechnol. 2003 May; 21(5):562-5 for methods), and nucleic acids encoding miR30 and GFP, as outlined in FIG. 1.

The complete sequence of the pFLIP insert is as follows:

[SEQ ID NO: 1]
gtcgacggat ccataacttc gtataggata ccttatacga agttatctca ggtaccgccaccatgaccga gtacaagccc
acggtgcgcc tcgccacccg cgacgacgtc cccagggccgtacgcaccct cgccgccgcg ttcgccgact accccgccac
gcgccacacc gtcgatccggaccgccacat cgagcgggtc accgagctgc aagaactctt cctcacgcgc
gtcgggctcgacatcggcaag gtgtgggtc gcggacgacg gcgccgcggt ggcggtctgg accacgccggagagcgtcga
agcgggggcg gtgttcgccg agatcggccc gcgcatggcc gagttgagcggttcccggct ggccgcgcag caacagatgg
aaggcctcct ggcgccgcac cggcccaaggagcccgcgtg gttcctggcc accgtcggcg tctcgcccga ccaccagggc
aagggtctgggcagcgccgt cgtgctcccc ggagtggagg cggccgagcg cgccggggtg cccgccttcctggagacctc
cgcgccccgc aacctcccct tctacgagcg gctcggcttc accgtcaccgccgacgtcga ggtgcccgaa ggaccgcgca
cctggtgcat gacccgcaag cccggtgccctgtacaagaa acagaaaatt gtggcaccag tgaaacagac tttgaatttt
gaccttctcaagttggcggg agacgtcgag tccaaccctg ggcccatgaa cccagccatc agcgtcgctctcctgctctc
agtcttgcag gtgtcccgag ggcagaaggt gaccagcctg acagcctgcctggtgaacca aaacctcgc ctggactgcc
gccatgagaa taacaccaag gataactccatccagcatga gttcagcctg acccgagaga agaggaagca cgtgctctca
ggcaccctcgggatacccga gcacacgtac cgctcccgcg tcaccctctc caaccagccc tatatcaaggtccttaccct
agccaacttc accaccaagg atgagggcga ctacttttgt gagcttcgagtctcgggcgc gaatcccatg agctccaata
aaagtatcag tgtgtataga gacaaactggtcaagtgtgg cggcataagc ctgctggttc agaacacatc ctggatgctg
ctgctgctgctttccctctc cctcctccaa gccctggact tcatttctct gtgatctaga agccataacttcgtatagta
cacattatac gaagttatgt ttaaacgcat tagtcttcca attgaaaaaagtgatttaat ttataccatt ttaattcagc
tttgtaaaaa tgtatcaaag agatagcaaggtattcagtt ttagtaaaca agataattgc tcctaaagta gccccttgaa
ttctggttgctcgagccttc tgttgggtta acctgaagaa gtaatcccag caagtgtttc caagatgtgcaggcaacgat
tctgtaaagt actgaagcct cattcaaaca tagtatatgt gctgccgaagcgagcactta acaaggcttg cggccgctac
ttgtacagct cgtccatgcc gagagtgatcccggcggcgg tcacgaactc cagcaggacc atgtgatcgc gcttctcgtt
ggggtctttgctcagggcgg actgggtgct caggtagtgg ttgtcgggca gcagcacggg gccgtcgccgatgggggtgt
tctgctggta gtggtcggcg agctgcacgc tgccgtcctc gatgttgtggcggatcttga agttcacctt gatgccgttc
ttctgcttgt cggccatgat atagacgttgtggctgttgt agttgtactc cagcttgtgc cccaggatgt tgccgtcctc
cttgaagtcgatgcccttca gctcgatgcg gttcaccagg gtgtcgccct cgaacttcac ctcggcgcgggtcttgtagt
tgccgtcgtc cttgaagaag atggtgcgct cctggacgta gccttcgggcatggcggact tgaagaagtc gtgctgcttc
atgtggtcgg ggtagcggct gaagcactgcacgccgtagg tcagggtggt cacgagggtg ggccagggca cgggcagctt
gccggtggtgcagatgaact tcagggtcag cttgccgtag gtggcatcgc cctcgccctc gccggacacgctgaacttgt
ggccgtttac gtcgccgtcc agctcgacca ggatgggcac caccccggtgaacagctcct cgcccttgct caccatggtg
gcgaccggta taacttcgta taaggtatcctatacgaagt tatccattca ggctgtgcta gcatcaatgg catggcacaa
agcttagccataacttcgta taatgtgtac tatacgaagt tatcccgggt taac.

The RNAi encoding sequence against firefly luciferase is as follows: AAGGTATATTGCTGTTGACAGTGAGCGAGCTCCCGTGAATTGGAATCCTAGTGAAGCC ACAGATGTAGGA′TTCCAATTCAGCGGGAGCCTGCCTACTGCCTCG

[SEQ ID NO: 2], and pFLIP comprising the RNAi sequence encoding sequence against firefly luciferase is as follows:

[SEQ ID NO: 3]
gtcgacggatccataacttcgtataggataccttatacgaagttatctcaggtaccgccaccatgaccgagtacaagcccacggtgcgcctcgcc
acccgcgacgacgtccccagggccgtacgcaccctcgccgccgcgttcgccgactaccccgccacgcgccacaccgtcgatccggaccgc
cacatcgagcgggtcaccgagctgcaagaactcttcctcacgcgcgtcgggctcgacatcggcaaggtgtgggtcgcggacgacggcgccg
cggtggcggtctggaccacgccggagagcgtcgaagcgggggcggtgttcgccgagatcggcccgcgcatggccgagttgagcggttccc
ggctggccgcgcagcaacagatggaaggcctcctggcgccgcaccggcccaaggagcccgcgtggttcctggccaccgtcggcgtctcgc
ccgaccaccagggcaagggtctgggcagcgccgtcgtgctccccggagtggaggcggccgagcgcgccggggtgcccgccttcctggag
acctccgcgccccgcaacctccccttctacgagcggctcggcttcaccgtcaccgccgacgtcgaggtgcccgaaggaccgcgcacctggtg
catgacccgcaagcccggtgccctgtacaagaaacagaaaattgtggcaccagtgaaacagactttgaattttgaccttctcaagttggcggga
gacgtcgagtccaaccctgggcccatgaacccagccatcagcgtcgctctcctgctctcagtcttgcaggtgtccgagggcagaaggtgacca
gcctgacagcctgcctggtgaaccaaaaccttcgcctggactgccgccatgagaataacaccaaggataactccatccagcatgagttcagcct
gacccgagagaagaggaagcacgtgctctcaggcaccctcgggatacccgagcacacgtaccgctcccgcgtcaccctctccaaccagccc
tatatcaaggtccttaccctagccaacttcaccaccaaggatgagggcgactacttttgtgagcttcgagtctcgggcgcgaatcccatgagctcc
aataaaagtatcagtgtgtatagagacaaactggtcaagtgtggcggcataagcctgctggttcagaacacatcctggatgctgctgctgctgctt
tccctctccctcctccaagccctggacttcatttctctgtgatctagaagccataacttcgtatagtacacattatacgaagttatgtttaaacgcatta
gtcttccaattgaaaaaagtgatttaatttataccattttaattcagctttgtaaaaatgtatcaaagagatagcaaggtattcagttttagtaaacaaga
taattgctcctaaagtagccccttgaattcCGAGGCAGTAGGCAGGCTCCCGCTGAATTGGAATCCTACAT
CTGTGGCTTCACTAGGATTCCAATTCACGGGAGCTCGCTCACTGTCAACAGCAATATAC
CTTctcgagccttctgttgggttaacctgaagaagtaatcccagcaagtgtttccaagatgtgcaggcaacgattctgtaaagtactgaagcctc
attcaaacatagtatatgtgctgccgaagcgagcacttaacaaggcttgcggccgctacttgtacagctcgtccatgccgagagtgatcccggcg
gcggtcacgaactccagcaggaccatgtgatcgcgcttctcgttggggtctttgctcagggcggactgggtgctcaggtagtggttgtcgggca
gcagcacggggccgtcgccgatgggggtgttctgctggtagtggtcggcgagctgcacgctgccgtcctcgatgttgtggcggatcttgaagtt
caccttgatgccgttcttctgcttgtcggccatgatatagacgttgtggctgttgtagttgtactccagcttgtgccccaggatgttgccgtcctccttg
aagtcgatgcccttcagctcgatgcggttcaccagggtgtcgccctcgaacttcacctcggcgcgggtcttgtagttgccgtcgtccttgaagaa
gatggtgcgctcctggacgtagccttcgggcatggcggacttgaagaagtcgtgctgcttcatgtggtcggggtagcggctgaagcactgcac
gccgtaggtcagggtggtcacgagggtgggccagggcacgggcagcttgccggtggtgcagatgaacttcagggtcagcttgccgtaggtg
gcatcgccctcgccctcgccggacacgctgaacttgtggccgtttacgtcgccgtccagctcgaccaggatgggcaccaccccggtgaacag
ctcctcgcccttgctcaccatggtggcgaccggtataacttcgtataaggtatcctatacgaagttatccattcaggctgtgctagcatcaatggcat
ggcacaaagcttagccataacttcgtataatgtgtactatacgaagttatcccgggttaac.

MSCV comprising the pFLIP insert with RNAi to Luciferase is as follows (FLIP insert in lowercase):

[SEQ ID NO: 4]
TGAAAGACCCCACCTGTAGGTTTGGCAAGCTAGCTTAAGTAACGCCATTTTGCAAGGC
ATGGAAAATACATAACTGAGAATAGAGAAGTTCAGATCAAGGTTAGGAACAGAGAGA
CAGCAGAATATGGGCCAAACAGGATATCTGTGGTAAGCAGTTCCTGCCCCGGCTCAGG
GCCAAGAACAGATGGTCCCCAGATGCGGTCCCGCCCTCAGCAGTTTCTAGAGAACCAT
CAGATGTTTCCAGGGTGCCCCAAGGACCTGAAAATGACCCTGTGCCTTATTTGAACTAA
CCAATCAGTTCGCTTCTCGCTTCTGTTCGCGCGCTTCTGCTCCCCGAGCTCAATAAAAG
AGCCCACAACCCCTCACTCGGCGCGCCAGTCCTCCGATAGACTGCGTCGCCCGGGTAC
CCGTATTCCCAATAAAGCCTCTTGCTGTTTGCATCCGAATCGTGGACTCGCTGATCCTT
GGGAGGGTCTCCTCAGATTGATTGACTGCCCACCTCGGGGGTCTTTCATTTGGAGGTTC
CACCGAGATTTGGAGACCCCTGCCTAGGGACCACCGACCCCCCCGCCGGGAGGTAAGC
TGGCCAGCGGTCGTTTCGTGTCTGTCTCTGTCTITGTGCGTGTTTGTGCCGGCATCTAAT
GTTTGCGCCTGCGTCTGTACTAGTTAGCTAACTAGCTCTGTATCTGGCGGACCCGTGGT
GGAACTGACGAGTTCTGAACACCCGGCCGCAACCCTGGGAGACGTCCCAGGGACTTTG
GGGGCCGTTTTTGTGGCCCGACCTGAGGAAGGGAGTCGATGTGGAATCCGACCCCGTC
AGGATATGTGGITCTGGTAGGAGACGAGAACCTAAAACAGTTCCCGCCTCCGTCTGAA
TTTTTGCTTTCGGTTTGGAACCGAAGCCGCGCGTCTTGTCTGCTGCAGCGCTGCAGCAT
CGTTCTGTGTTGTCTCTGTCTGACTGTGTTTCTGTATTTGTCTGAAAATTAGGGCCAGAC
TGTTACCACTCCCTTAAGTTTGACCTTAGGTCACTGGAAAGATGTCGAGCGGATCGCTC
ACAACCAGTCGGTAGATGTCAAGAAGAGACGTTGGGTTACCTTCTGCTCTGCAGAATG
GCCAACCTITAACGTCGGATGGCCGCGAGACGGCACCTTTAACCGAGACCTCATCACC
CAGGTTAAGATCAAGGTCTTTTCACCTGGCCCGCATGGACACCCAGACCAGGTCCCCT
ACATCGTGACCTGGGAAGCCTTGGCTTTTGACCCCCCTCCCTGGGTCAAGCCCTTTGTA
CACCCTAAGCCTCCGCCTCCTCTTCCTCCATCCGCCCCGTCTCTCCCCCTTGAACCTCCT
CGTTCGACCCCGCCTCGATCCTCCCTTTATCCAGCCCTCACTCCTTCTCTAGGCGCCGG
AATTAGAtccataacttcgtataggataccttatacgaagttatctcaggtaccgccaccatgaccgagtacaagcccacggtgcgcctcg
ccacccgcgacgacgtccccagggccgtacgcaccctcgccgccgcgttcgccgactaccccgccacgcgccacaccgtcgatccggacc
gccacatcgagcgggtcaccgagctgcaagaactcttcctcacgcgcgtcgggctcgacatcggcaaggtgtgggtcgcggacgacggcgc
cgcggtggcggtctggaccacgccggagagcgtcgaagcgggggcggtgttcgccgagatcggcccgcgcatggccgagttgagcggttc
ccggctggccgcgcagcaacagatggaaggcctcctggcgccgcaccggcccaaggagcccgcgtggttcctggccaccgtcggcgtctc
gcccgaccaccagggcaagggtctgggcagcgccgtcgtgctccccggagtggaggcggccgagcgcgccggggtgcccgccttcctgg
agacctccgcgccccgcaacctccccttctacgagcggctcggcttcaccgtcaccgccgacgtcgaggtgcccgaaggaccgcgcacctg
gtgcatgacccgcaagcccggtgccctgtacaagaaacagaaaattgtggcaccagtgaaacagactttgaattttgaccttctcaagttggcgg
gagacgtcgagtccaaccctgggcccatgaacccagccatcagcgtcgctctcctgctctcagtcttgcaggtgtcccgagggcagaaggtga
ccagcctgacagcctgcctggtgaaccaaaaccttcgcctggactgccgccatgagaataacaccaaggataactccatccagcatgagttca
gcctgacccgagagaagaggaagcacgtgctctcaggcaccctCgggatacccgagcacacgtaccgctcccgcgtcaccctctccaacca
gccctatatcaaggtccttaccctagccaacttcaccaccaaggatgagggcgactacttttgtgagcttcgagtctcgggcgcgaatcccatga
gctccaataaaagtatcagtgtgtatagagacaaactggtcaagtgtggcggcataagcctgctggttcagaacacatcctggatgctgctgctg
ctgctttccctctccctcctccaagccctggacttcatttctctgtgatctagaagccataacttcgtatagtacacattatacgaagttatgtttaaacg
cattagtcttccaattgaaaaaagtgatttaatttataccattttaattcagctttgtaaaaatgtatcaaagagatagcaaggtattcagttttagtaaac
aagataattgctcctaaagtagccccttgaattcCGAGGCAGTAGGCAGGCTCCCGCTGAATTGGAATCCTA
CATCTGTGGCTTCACTAGGATTCCAATTCACGGGAGCTCGCTCACTGTCAACAGCAATA
TACCTTctcgagccttctgttgggttaacctgaagaagtaatcccagcaagtgtttccaagatgtgcaggcaacgattctgtaaagtactgaa
gcctcattcaaacatagtatatgtgctgccgaagcgagcacttaacaaggcttgcggccgctacttgtacagctcgtccatgccgagagtgatcc
cggcggcggtcacgaactccagcaggaccatgtgatcgcgcttctcgttggggtctttgctcagggcggactgggtgctcaggtagtggttgtc
gggcagcagcacggggccgtcgccgatgggggtgttctgctggtagtggtcggcgagctgcacgctgccgtcctcgatgttgtggcggatctt
gaagttcaccttgatgccgttcttctgcttgtcggccatgatatagacgttgtggctgttgtagttgtactccagcttgtgccccaggatgttgccgtc
ctccttgaagtcgatgcccttcagctcgatgcggttcaccagggtgtcgccctcgaacttcacctcggcgcgggtcttgtagttgccgtcgtccttg
aagaagatggtgcgctcctggacgtagccttcgggcatggcggacttgaagaagtcgtgctgcttcatgtggtcggggtagcggctgaagcac
tgcacgccgtaggtcagggtggtcacgagggtgggccagggcacgggcagcttgccggtggtgcagatgaacttcagggtcagcttgccgt
aggtggcatcgccctcgccctcgccggacacgctgaacttgtggccgtttacgtcgccgtccagctcgaccaggatgggcaccaccccggtg
aacagctcctcgcccttgctcaccatggtggcgaccggtataacttcgtataaggtatcctatacgaagttatccattcaggctgtgctagcatcaa
tggcatggcacaaagcttagccataacttcgtataatgtgtactatacgaagttatcccgggttAAACGACCTGCAGCCAAGCT
TATCGATAAAATAAAAGATTTTATTTAGTCTCCAGAAAAAGGGGGGAATGAAAGACCC
CACCTGTAGGTTTGGCAAGCTAGCTTAAGTAACGCCATTTTGCAAGGCATGGAAAATA
CATAACTGAGAATAGAGAAGTTCAGATCAAGGTTAGGAACAGAGAGACAGCAGAATA
TGGGCCAAACAGGATATCTGTGGTAAGCAGTTCCTGCCCCGGCTCAGGGCCAAGAACA
GATGGTCCCCAGATGCGGTCCCGCCCTCAGCAGTTTCTAGAGAACCATCAGATGTTTCC
AGGGTGCCCCAAGGACCTGAAAATGACCCTGTGCCTTATTTGAACTAACCAATCAGTT
CGCTTCTCGCTTCTGTTCGCGCGCTTCTGCTCCCCGAGCTCAATAAAAGAGCCCACAAC
CCCTCACTCGGCGCGCCAGTCCTCCGATAGACTGCGTCGCCCGGGTACCCGTGTATCCA
ATAAACCCTCTTGCAGTTGCATCCGACTTGTGGTCTCGCTGTTCCTTGGGAGGGTCTCC
TCTGAGTGATTGACTACCCGTCAGCGGGGGTCTTTCATGGGTAACAGTTTCTTGAAGTT
GGAGAACAACATTCTGAGGGTAGGAGTCGAATATTAAGTAATCCTGACTCAATTAGCC
ACTGTTTTGAATCCACATACTCCAATACTCCTGAAATAGTTCATTATGGACAGCGCAGA
AGAGCTGGGGAGAATTAATTCGTAATCATGGTCATAGCTGTTTCCTGTGTGAAATTGTT
ATCCGCTCACAATTCCACACAACATACGAGCCGGAAGCATAAAGTGTAAAGCCTGGGG
TGCCTAATGAGTGAGCTAACTCACATTAATTGCGTTGCGCTCACTGCCCGCTTTCCAGT
CGGGAAACCTGTCGTGCCAGCTGCATTAATGAATCGGCCAACGCGCGGGGAGAGGCG
GTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATC
AGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGGA
CAGTATTTGGTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAA
GGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACGCTCA
CCGGCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTAGAGT
AAGTAGTTCGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAA
GTCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCTT
CGGGGCGAAAACTCTCAAGGATCTTACCGCTGTTGAGATCCAGTTCGATGTAACCCAC
TCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTCTCATG
AGCGGATACATATTTGAATGTATTTAGAAAAATAAACAAATAGGGGTTCCGCGCACAT
TTCCCCGAAAAGTGCCACCTGACGTCTAAGAAACCATTATTATCATGACATTAACCTAT
AAAAATAGGCGTATCACGAGGCCCTITCGTCTCGCGCGTTTCGGTGATGACGGTGAAA
ACCTCTGACACATGCAGCTCCCGGAGACGGTCACAGCTTGTCTGTAAGCGGATGCCGG
GAGCAGACAAGCCCGTCAGGGCGCGTCAGCGGGTGTTGGCGGGTGTCGGGGCTGGCTT
AACTATGCGGCATCAGAGCAGATTGTACTGAGAGTGCACCATATGCGGTGTGAAATAC
CGCACAGATGCGTAAGGAGAAAATACCGCATCAGGCGCCATTCGCCATTCAGGCTGCG
CAACTGTTGGGAAGGGCGATCGGTGCGGGCCTCTTCGCTATTACGCCAGCTGGCGAAA
GGGGGATGTGCTGCAAGGCGATTAAGTTGGGTAACGCCAGGGTTTTCCCAGTCACGAC
GTTGTAAAACGACGGCGCAAGGAAGCAGCCCAGTAGTAGGTTGAGGCCGTTGAGCAC
CGCCGCCGCAAGGAATGGTGCATGCAAGGAGATGGCGCCCAACAGTCCCCCGGCCAC
GGGGCCTGCCACCATACCCACGCCGAAACAAGCGCTCATGAGCCCGAAGTGGCGAGC
CCGATCTTCCCCATCGGTGATGTCGGCGATATAGGCGCCAGCAACCGCACCTGTGGCG
CCGGTGATGCCGGCCACGATGCGTCCGGCGTAGAGGCGATTAGTCCAATTTGTTAAAG
ACAGGATATCAGTGGTCCAGGCTCTAGTTTTGACTCAACAATATCACCAGCTGAAGCCT
ATAGAGTACGAGCCATAGATAAAATAAAAGATTTTATTTAGTCTCCAGAAAAAGGGGG
GAA.

The RNAi encoding sequence against p53 is as follows:

[SEQ ID NO: 5]
AAGGTATATTGCTGTTGACAGTGAGCGCCCACTACAAGTACATGTGTAA
TGTGAAGCCACAGATGTATTACACATGTACTTGTAGTGGATGCCTACTG
CCTCG.

The pFLIP insert comprising an RNAi to p53 has a nucleic acid sequence as follows (microRNA-short hairpin to p53 in upper case):

[SEQ ID NO: 6]
tccataacttcgtataggataccttatacgaagttatctcaggtaccgccaccatgaccgagtacaagcccacggtgcgcctcgccacccgcgac
gacgtccccagggccgtacgcaccctcgccgccgcgttcgccgactaccccgccacgcgccacaccgtcgatccggaccgccacatcgagc
gggtcaccgagctgcaagaactcttcctcacgcgcgtcgggctcgacatcggcaaggtgtgggtcgcggacgacggcgccgcggtggcggt
ctggaccacgccggagagcgtcgaagcgggggcggtgttcgccgagatcggcccgcgcatggccgagttgagcggttcccggctggccgc
gcagcaacagatggaaggcctcctggcgccgcaccggcccaaggagcccgcgtggttcctggccaccgtcggcgtctcgcccgaccacca
gggcaagggtctgggcagcgccgtcgtgctccccggagtggaggcggccgagcgcgccggggtgcccgccttcctggagacctccgcgcc
ccgcaacctccccttctacgagcggctcggcttcaccgtcaccgccgacgtcgaggtgcccgaaggaccgcgcacctggtgcatgacccgca
agcccggtgccctgtacaagaaacagaaaattgtggcaccagtgaaacagactttgaattttgaccttctcaagttggcgggagacgtcgagtcc
aaccctgggcccatgaacccagccatcagcgtcgctctcctgctctcagtcttgcaggtgtcccgagggcagaaggtgaccagcctgacagcct
gcctggtgaaccaaaaccttcgcctggactgccgccatgagaataacaccaaggataactccatccagcatgagttcagcctgacccgagaga
agaggaagcacgtgctctcaggcaccctcgggatacccgagcacacgtaccgctcccgcgtcaccctctccaaccagccctatatcaaggtcc
ttaccctagccaacttcaccaccaaggatgagggcgactacttttgtgagcttcgagtctcgggcgcgaatcccatgagctccaataaaagtatca
gtgtgtatagagacaaactggtcaagtgtggcggcataagcctgctggttcagaacacatcctggatgctgctgctgctgctttccctctccctcct
ccaagccctggacttcatttctctgtgatctagaagccataacttcgtatagtacacattatacgaagttatGTTTAAACGCATTAGTC
TTCCAATTGAAAAAAGTGATTTAATTTATACCATTTTAATTCAGCTTTGTAAAAATGTAT
CAAAGAGATAGCAAGGTATTCAGTTTTAGTAAACAAGATAATTGCTCCTAAAGTAGCC
CCTTGAATTCCGAGGCAGTAGGCATCCACTACAAGTACATGTGTAATACATCTGTGGCT
TCACTATTACACATGTACTTGTAGTGGGCGCTCACTGTCAACAGCAATATACCTTCTCG
AGCCTTCTGTTGGGTTAACCTGAAGAAGTAATCCCAGCAAGTGTTTCCAAGATGTGCAG
GCAACGATTCTGTAAAGTACTGAAGCCTCATTCAAACATAGTATATGTGCTGCCGAAGC
GAGCACTTAACAAGGCTTGCGGCCGCtacttgtacagctcgtccatgccgagagtgatcccggcggcggtcacgaact
ccagcaggaccatgtgatcgcgcttctcgttggggtctttgctcagggcggactgggtgctcaggtagtggttgtcgggcagcagcacggggcc
gtcgccgatgggggtgttctgctggtagtggtcggcgagctgcacgctgccgtcctcgatgttgtggcggatcttgaagttcaccttgatgccgtt
cttctgcttgtcggccatgatatagacgttgtggctgttgtagttgtactccagcttgtgccccaggatgttgccgtcctccttgaagtcgatgcccttc
agctcgatgcggttcaccagggtgtcgccctcgaacttcacctcggcgcgggtcttgtagttgccgtcgtccttgaagaagatggtgcgctcctg
gacgtagccttcgggcatggcggacttgaagaagtcgtgctgcttcatgtggtcggggtagcggctgaagcactgcacgccgtaggtcagggt
ggtcacgagggtgggccagggcacgggcagcttgccggtggtgcagatgaacttcagggtcagcttgccgtaggtggcatcgccctcgccct
cgccggacacgctgaacttgtggccgtttacgtcgccgtccagctcgaccaggatgggcaccaccccggtgaacagctcctcgcccttgctcac
catggtggcgaccggtataacttcgtataaggtatcctatacgaagttatccattcaggctgtgctagcatcaatggcatggcacaaagcttagcca
taacttcgtataatgtgtactatacgaagttatcccgggtt.

The MSCV FLIP-p53 construct has a nucleotide sequence as follows (FLIP insert begins base 1411 lower case, microRNA-short hairpin to p53 in upper case):

[SEQ ID NO: 7]
TGAAAGACCCCACCTGTAGGTTTGGCAAGCTAGCTTAAGTAACGCCATTTTGCAAGGCA
TGGAAAATACATAACTGAGAATAGAGAAGTTCAGATCAAGGTTAGGAACAGAGAGAC
AGCAGAATATGGGCCAAACAGGATATCTGTGGTAAGCAGTTCCTGCCCCGGCTCAGGG
CCAAGAACAGATGGTCCCCAGATGCGGTCCCGCCCTCAGCAGTTTCTAGAGAACCATC
AGATGTTTCCAGGGTGCCCCAAGGACCTGAAAATGACCCTGTGCCTTATTTGAACTAAC
CAATCAGTTCGCTTCTCGCTTCTGTTCGCGCGCTTCTGCTCCCCGAGCTCAATAAAAGA
GCCCACAACCCCTCACTCGGCGCGCCAGTCCTCCGATAGACTGCGTCGCCCGGGTACCC
GTATTCCCAATAAAGCCTCTTGCTGTTTGCATCCGAATCGTGGACTCGCTGATCCTTGG
GAGGGTCTCCTCAGATTGATTGACTGCCCACCTCGGGGGTCTTTCATTTGGAGGTTCCA
CCGAGATTTGGAGACCCCTGCCTAGGGACCACCGACCCCCCCGCCGGGAGGTAAGCTG
GCCAGCGGTCGTTTCGTGTCTGTCTCTGTCTTTGTGCGTGTTTGTGCCGGCATCTAATGT
TTGCGCCTGCGTCTGTACTAGTTAGCTAACTAGCTCTGTATCTGGCGGACCCGTGGTGG
AACTGACGAGTTCTGAACACCCGGCCGCAACCCTGGGAGACGTCCCAGGGACTTTGGG
GGCCGTTTTTGTGGCCCGACCTGAGGAAGGGAGTCGATGTGGAATCCGACCCCGTCAG
GATATGTGGTTCTGGTAGGAGACGAGAACCTAAAACAGTTCCCGCCTCCGTCTGAATTT
TTGCTTTCGGTTTGGAACCGAAGCCGCGCGTCTTGTCTGCTGCAGCGCTGCAGCATCGT
TCTGTGTTGTCTCTGTCTGACTGTGTTTCTGTATTTGTCTGAAAATTAGGGCCAGACTGT
TACCACTCCCTTAAGTTTGACCTTAGGTCACTGGAAAGATGTCGAGCGGATCGCTCACA
ACCAGTCGGTAGATGTCAAGAAGAGACGTTGGGTTACCTTCTGCTCTGCAGAATGGCC
AACCTTTAACGTCGGATGGCCGCGAGACGGCACCTTTAACCGAGACCTCATCACCCAG
GTTAAGATCAAGGTCTTTTCACCTGGCCCGCATGGACACCCAGACCAGGTCCCCTACAT
CGTGACCTGGGAAGCCTTGGCTTTTGACCCCCCTCCCTGGGTCAAGCCCTTTGTACACC
CTAAGCCTCCGCCTCCTCTTCCTCCATCCGCCCCGTCTCTCCCCCTTGAACCTCCTCGTT
CGACCCCGCCTCGATCCTCCCTTTATCCAGCCCTCACTCCTTCTCTAGGCGCCGGAATTA
GAtccataacttcgtataggataccttatacgaagttatctcaggtaccgccaccatgaccgagtacaagcccacggtgcgcctcgccacccgc
gacgacgtccccagggccgtacgcaccctcgccgccgcgttcgccgactaccccgccacgcgccacaccgtcgatccggaccgccacatcg
agcgggtcaccgagctgcaagaactcttcctcacgcgcgtcgggctcgacatcggcaaggtgtgggtcgcggacgacggcgccgcggtggc
ggtctggaccacgccggagagcgtcgaagcgggggcggtgttcgccgagatcggcccgcgcatggccgagttgagcggttcccggctggc
cgcgcagcaacagatggaaggcctcctggcgccgcaccggcccaaggagcccgcgtggttcctggccaccgtcggcgtctcgcccgacca
ccagggcaagggtctgggcagcgccgtcgtgctccccggagtggaggcggccgagcgcgccggggtgcccgccttcctggagacctccgc
gccccgcaacctccccttctacgagcggctcggcttcaccgtcaccgccgacgtcgaggtgcccgaaggaccgcgcacctggtgcatgaccc
gcaagcccggtgccctgtacaagaaacagaaaattgtggcaccagtgaaacagactttgaattttgaccttctcaagttggcgggagacgtcgag
tccaaccctgggcccatgaacccagccatcagcgtcgctctcctgctctcagtcttgcaggtgtcccgagggcagaaggtgaccagcctgacag
cctgcctggtgaaccaaaaccttcgcctggactgccgccatgagaataacaccaaggataactccatccagcatgagttcagcctgacccgaga
gaagaggaagcacgtgctctcaggcaccctcgggatacccgagcacacgtaccgctcccgcgtcaccctctccaaccagccctatatcaaggt
ccttaccctagccaacttcaccaccaaggatgagggcgactacttttgtgagcttcgagtctcgggcgcgaatcccatgagctccaataaaagtat
cagtgtgtatagagacaaactggtcaagtgtggcggcataagcctgctggttcagaacacatcctggatgctgctgctgctgctttccctctccctc
ctccaagccctggacttcatttctctgtgatctagaagccataacttcgtatagtacacattatacgaagttatGTTTAAACGCATTAGT
CTTCCAATTGAAAAAAGTGATTTAATTTATACCATTTTAATTCAGCTTTGTAAAAATGTA
TCAAAGAGATAGCAAGGTATTCAGTTTTAGTAAACAAGATAATTGCTCCTAAAGTAGC
CCCTTGAATTCCGAGGCAGTAGGCATCCACTACAAGTACATGTGTAATACATCTGTGGC
TTCACTATTACACATGTACTTGTAGTGGGCGCTCACTGTCAACAGCAATATACCTTCTC
GAGCCTTCTGTTGGGTTAACCTGAAGAAGTAATCCCAGCAAGTGTTTCCAAGATGTGCA
GGCAACGATTCTGTAAAGTACTGAAGCCTCATTCAAACATAGTATATGTGCTGCCGAAG
CGAGCACTTAACAAGGCTTGCGGCCGCtacttgtacagctcgtccatgccgagagtgatcccggcggcggtcacgaa
ctccagcaggaccatgtgatcgcgcttctcgttggggtctttgctcagggcggactgggtgctcaggtagtggttgtcgggcagcagcacgggg
ccgtcgccgatgggggtgttctgctggtagtggtcggcgagctgcacgctgccgtcctcgatgttgtggcggatcttgaagttcaccttgatgccg
ttcttctgcttgtcggccatgatatagacgttgtggctgttgtagttgtactccagcttgtgccccaggatgttgccgtcctccttgaagtcgatgccctt
cagctcgatgcggttcaccagggtgtcgccctcgaacttcacctcggcgcgggtcttgtagttgccgtcgtccttgaagaagatggtgcgctcctg
gacgtagccttcgggcatggcggacttgaagaagtcgtgctgcttcatgtggtcggggtagcggctgaagcactgcacgccgtaggtcagggt
ggtcacgagggtgggccagggcacgggcagcttgccggtggtgcagatgaacttcagggtcagcttgccgtaggtggcatcgccctcgccct
cgccggacacgctgaacttgtggccgtttacgtcgccgtccagctcgaccaggatgggcaccaccccggtgaacagctcctcgcccttgctcac
catggtggcgaccggtataacttcgtataaggtatcctatacgaagttatccattcaggctgtgctagcatcaatggcatggcacaaagcttagc
cataacttcgtataatgtgtactatacgaagttatcccgggttAAACGACCTGCAGCCAAGCTTATCGATAAAATA
AAAGATTTTATTTAGTCTCCAGAAAAAGGGGGGAATGAAAGACCCCACCTGTAGGTTT
GGCAAGCTAGCTTAAGTAACGCCATTTTGCAAGGCATGGAAAATACATAACTGAGAAT
AGAGAAGTTCAGATCAAGGTTAGGAACAGAGAGACAGCAGAATATGGGCCAAACAGG
ATATCTGTGGTAAGCAGTTCCTGCCCCGGCTCAGGGCCAAGAACAGATGGTCCCCAGA
TGCGGTCCCGCCCTCAGCAGTTTCTAGAGAACCATCAGATGTTTCCAGGGTGCCCCAAG
GACCTGAAAATGACCCTGTGCCTTATTTGAACTAACCAATCAGTTCGCTTCTCGCTTCTG
TTCGCGCGCTTCTGCTCCCCGAGCTCAATAAAAGAGCCCAGAACCCCTCACTCGGCGCG
CCAGTCCTCCGATAGACTGCGTCGCCCGGGTACCCGTGTATCCAATAAACCCTCTTGCA
GTTGCATCCGACTTGTGGTCTCGCTGTTCCTTGGGAGGGTCTCCTCTGAGTGATTGACTA
CCCGTCAGCGGGGGTCTTTCATGGGTAACAGTTTCTTGAAGTTGGAGAACAACATTCTG
AGGGTAGGAGTCGAATATTAAGTAATCCTGACTCAATTAGCCACTGTTTTGAATCCACA
TACTCCAATACTCCTGAAATAGTTCATTATGGACAGCGCAGAAGAGCTGGGGAGAATT
AATTCGTAATCATGGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAA
AGAACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTT
CTTGAAGTGGTGGCCTAACTACGGCTACACTAGAAGGACAGTATTTGGTATCTGCGCTC
TGCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAAA
GGATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCC
ATAGTTGCCTGACTCCCCGTCGTGTAGATAACTACGATACGGGAGGGCTTACCATCTGG
CCCCAGTGCTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAA
GATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAA
AAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGA
AAAATAAACAAATAGGGGTTCCGCGCACATTTCCCCGAAAAGTGCCACCTGACGTCTA
AGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGCGTCA
GCGGGTGTTGGCGGGTGTCGGGGCTGGCTTAACTATGCGGCATCAGAGCAGATTGTAC
TGAGAGTGCACCATATGCGGTGTGAAATACCGCACAGATGCGTAAGGAGAAAATACCG
CATCAGGCGCCATTCGCCATTCAGGCTGCGCAACTGTTGGGAAGGGCGATCGGTGCGG
GCCTCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCGCAAGGAAGCAG
CCCAGTAGTAGGTTGAGGCCGTTGAGCACCGCCGCCGCAAGGAATGGTGCATGCAAGG
AGATGGCGCCCAACAGTCCCCCGGCCACGGGGCCTGCCACCATACCCACGCCGAAACA
AGCGCTCATGAGCCCGAAGTGGCGAGCCCGATCTTCCCCATCGGTGATGTCGGCGATAT
AGGCGCCAGCAACCGCACCTGTGGCGCCGGTGATGCCGGCCACGATGCGTCCGGCGTA
GAGGCGATTAGTCCAATTTGTTAAAGACAGGATATCAGTGGTCCAGGCTCTAGTTTTGA
CTCAACAATATCACCAGCTGAAGCCTATAGAGTACGAGCCATAGATAAAATAAAAGAT
TTTATTTAGTCTCCAGAAAAAGGGGGGAA.

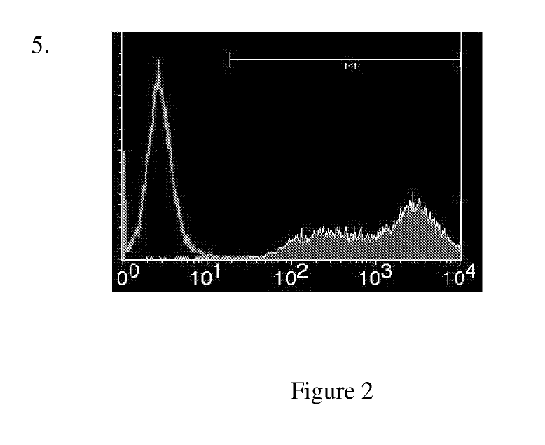

Another FLIP insert comprising loxP 5171 and loxP 2272 genes, a modified puromycin resistance cassette incorporating the foot-and-mouth-disease virus (FMDV) 2A encoding sequence at its C-terminus, fused in frame to a gene encoding GFP, and nucleic acids encoding miR30 and GFP, was similarly constructed and is outlined in FIG. 2.

The complete sequence of this construct, pPRIME-201 with puro2A-GFP insert, is as follows (upper case bases 3210-4628):

[SEQ ID NO: 8]
gtcgacggatcgggagatctcccgatcccctatggtgcactctcagtacaatctgctctgatgccgcatagttaagccagtatctgctccctgcttg
tgtgttggaggtcgctgagtagtgcgcgagcaaaatttaagctacaacaaggcaaggcttgaccgacaattgcatgaagaatctgcttagggtta
ggcgttttgcgctgcttcgcgatgtacgggccagatatacgcgttgacattgattattgactagttattaatagtaatcaattacggggtcattagttc
atagcccatatatggagttccgcgttacataacttacggtaaatggcccgcctggctgaccgcccaacgacccccgcccattgacgtcaataatg
acgtatgttcccatagtaacgccaatagggactttccattgacgtcaatgggtggagtatttacggtaaactgcccacttggcagtacatcaagtgt
atcatatgccaagtacgccccctattgacgtcaatgacggtaaatggcccgcctggcattatgcccagtacatgaccttatgggactttcctacttg
gcagtacatctacgtattagtcatcgctattaccatggtgatgcggttttggcagtacatcaatgggcgtggatagcggtttgactcacggggatttc
caagtctccaccccattgacgtcaatgggagtttgttttggcaccaaaatcaacgggactttccaaaatgtcgtaacaactccgccccattgacgc
aaatgggcggtaggcgtgtacggtgggaggtctatagaccagatctgagcctgggagctctctggctaactagggaacccactgcttaagcct
caataaagcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctgaagcgc
gcacggcaagaggcgaggggcggcgactggtgagtacgccaaaaattttgactagcggaggctagaaggagagagatgggtgcgagagc
gtcagtattaagcgggggagaattagatcgcgatgggaaaaaattcggttaaggccagggggaaagaaaaaatataaataaacatatagtatg
ggcaagcagggagctagaacgattcgcagttaatcctggcctgttagaaacatcagaaggctgtagacaaatactgggacagctacaaccatc
ccttcagacaggatcagaagaacttagatcattatataatacagtagcaaccctctattgtgtgcatcaaaggatagagataaaagacaccaagg
aagctttagacaagatagaggaagagcaaaacaaaagtaagaccaccgcacagcaagcggccggccgcgctgatcttcagacctggagga
ggagatatgagggacaattggagaagtgaattatataaatataaagtagtaaaaattgaaccattaggagtagcacccaccaaggcaaagagaa
gagtggtgcagagagaaaaaagagcagtgggaataggagctttgttccttgggttcttgggagcagcaggaagcactatgggcgcagcgtca
atgacgctgacggtacaggccagacaattattgtctggtatagtgcagcagcagaacaatttgctgagggctattgaggcgcaacagcatctgtt
gcaactcacagtctggggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagatttggaatcacacgacctggatg
gagtgggacagagaaattaacaattacacaagcttaatacactccttaattgaagaatcgcaaaaccagcaagaaaagaatgaacaagaattatt
ggaattagataaatgggcaagtttgtggaattggtttaacataacaaattggctgtggtatataaaattattcataatgatagtaggaggcttggtag
gtttaagaatagtttttgctgtactttctatagtgaatagagttaggcagggatattcaccattatcgtttcagacccacctcccaaccccgagggga
cccgacaggcccgaaggaatagaagaagaaggtggagagagagacagagacagatccattcgattagtgaacggatcggcactgcgtgcg
ccaattctgcagacaaatggcagtattcatccacaattttaaaagaaaaggggggattggggggtacagtgcaggggaaagaatagtagacata
atagcaacagacatacaaactaaagaattacaaaaacaaattacaaaaattcaaaattttcgggtttattacagggacagcagagatccagtttggt
tagtaccgggcccgctctagacgtattaccgccatgcattagttattaatagtaatcaattacggggtcattagttcatagcccatatatggagttcc
gcgttacataacttacggtaaatggcccgcctggctgaccgcccaacgacccccgcccattgacgtcaataatgacgtatgttcccatagtaacg
ccaatagggactttccattgacgtcaatgggtggagtatttacggtaaactgcccacttggcagtacatcaagtgtatcatatgccaagtacgccc
cctattgacgtcaatgacggtaaatggcccgcctggcattatgcccagtacatgaccttatgggactttcctacttggcagtacatctacgtattagt
catcgctattaccatggtgatgcggttttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgcaaatgggcggtaggcgtgt
acggtgggaggtctatataagcagagctggtttagtgaaccgtcagatccgctagcgctaccggtGCCACCATGGTGGAGTAC
AAGCCCACGGTGCGCCTCGCCACCCGCGACGACGTCCCCAGGGCCGTACGCACCCTCG
CCGCCGCGTTCGCCGACTACCCCGCCACGCGCCACACCGTCGATCCGGACCGCCACAT
CGAGCGGGTCACCGAGCTGCAAGAACTCTTCCTCACGCGCGTCGGGCTCGACATCGGC
AAGGTGTGGGTCGCGGACGACGGCGCCGCGGTGGCGGTCTGGACCACGCCGGAGA
GCGTCGAAGCGGGGGCGGTGTTCGCCGAGATCGGCCCGCGCATGGCCGAGTTGAGCG
GTTCCCGGCTGGCCGCGCAGCAACAGATGGAAGGCCTCCTGGCGCCGCACCGGCCCAA
GGAGCCCGCGTGGTTCCTGGCCACCGTCGGCGTCTCGCCCGACCACCAGGGCAAGGGT
CTGGGCAGCGCCGTCGTGCTCCCCGGAGTGGAGGCGGCCGAGCGCGCCGGGGTGCCCG
CCTTCCTGGAGACCTCCGCGCCCCGCAACCTCCCCTTCTACGAGCGGCTCGGCTTCACC
GTCACCGCCGACGTCGAGGTGCCCGAAGGACCGCGCACCTGGTGCATGACCCGCAAGC
CCGGTGCCAAACAGAAAATTGTGGCACCAGTGAAACAGACTTTGAATTTTGACCTTCT
CAAGTTGGCGGGAGACGTCGAGTCCAACCCTGGGCCCGGCCCGGTCGCCACCATGGTG
AGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACG
GCAAGCTGACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCGACCACATG
AAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGA
CACCCTGGTGAACCGCATCGAGCTGAAGGGCATCGACTTCAAGGAGGACGGCAACATC
CTGGGGCACAAGCTGGAGTACAACTACAACAGCCACAACGTCTATATCATGGCCGACA
AGCAGAAGAACGGCATCAAGGTGAACTTCAAGATCCGCCACAACATCGAGGACGGCA
GCGTGCAGCTCGCCGACCACTACCAGCAGAACACCCCCATCGGCGACGGCCCCGTGCT
GCTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCA
TGGACGAGCTGTACAAGTAGcggccgcaagccttgttaagtgctcgcttcggcagcacatatactatgtttgaatgaggcttc
agtactttacagaatcgttgcctgcacatcttggaaacacttgctgggattacttcttcaggttaacccaacagaaggctcgagcaaccagaattca
aggggctactttaggagcaattatcttgtttactaaaactgaataccttgctatctctttgatacatttttacaaagctgaattaaaatggtataaattaaa
tcacttttttcaattggaagactaatgcgtttaaacacgcggcgacgcgttcgaccgaataaaacctgtgacggaagatcacttcgcagaataaat
aaatcctggtgtccctgttgataccgggaagccctgggccaacttttggcgaaaatgagacgttgatcggcacgtaagaggttccaactttcacc
ataatgaaataagatcactaccgggcgtattttttgagttgtcgagattttcaggagctaaggaagctaaaatggagaaaaaaatcactggatatac
caccgttgatatatcccaatggcatcgtaaagaacattttgaggcatttcagtcagttgctcaatgtacctataaccagaccgttcagctggatatta
cggcctttttaaagaccgtaaagaaaaataagcacaagttttatccggcctttattcacattcttgcccgcctgatgaatgctcatccggaattacgta
tggcaatgaaagacggtgagctggtgatatgggatagtgttcacccttgttacaccgttttccatgagcaaactgaaacgttttcatcgctctggag
tgaataccacgacgatttccggcagtttctacacatatattcgcaagatgtggcgtgttacggtgaaaacctggcctatttccctaaagggtttattg
agaatatgtttttcgtctcagccaatccctgggtgagtttcaccagttttgatttaaacgtggccaatatggacaacttcttcgcccccgttttcaccat
gggcaaatattatacgcaaggcgacaaggtgctgatgccgctggcgattcaggttcatcatgccgtttgtgatggcttccatgtcggcagaatgct
taatgaattacaacagtactgcgatgagtggcagggcggggcgtaatttttttaaggcagttattggtgcccttaaacgcctggttgctacgcctga
ataagtgataataagcggatgaatggcagaaattcggatctcgaccgcgtttgggcggtggctccctgccacgcggctccgaacagaagctga
tctccgaagaggatctgacatgtgtttaaacctcgacttaattaagtcgagggtcgacggtatcgataagctcgcttcacgagatcatgtttaaggg
ttccggttccactaggtacaattcgatatcaagcttatcgataatcaacctctggattacaaaatttgtgaaagattgactggtattcttaactatgttgc
tccttttacgctatgtggatacgctgctttaatgcctttgtatcatgctattgcttcccgtatggctttcattttctcctccttgtataaatcctggttgctgtc
tctttatgaggagttgtggcccgttgtcaggcaacgtggcgtggtgtgcactgtgtttgctgacgcaacccccactggttggggcattgccaccac
ctgtcagctcctttccgggactttcgctttccccctccctattgccacggcggaactcatcgccgcctgccttgcccgctgctggacaggggctcg
gctgttgggcactgacaattccgtggtgttgtcggggaaatcatcgtcctttccttggctgctcgcctgtgttgccacctggattctgcgcgggacg
tccttctgctacgtcccttcggccctcaatccagcggaccttccttcccgcggcctgctgccggctctgcggcctcttccgcgtcttcgccttcgcc
ctcagacgagtcggatctccctttgggccgcctccccgcatcgataccgtcgacctcgatcgagacctagaaaaacatggagcaatcacaagta
gcaatacagcagctaccaatgctgattgtgcctggctagaagcacaagaggaggaggaggtgggttttccagtcacacctcaggtacctttaag
accaatgacttacaaggcagctgtagatcttagccactttttaaaagaaaaggggggactggaagggctaattcactcccaacgaagacaagat
atccttgatctgtggatctaccacacacaaggctacttccctgattggcagaactacacaccagggccagggatcagatatccactgacctttgga
tggtgctacaagctagtaccagttgagcaagagaaggtagaagaagccaatgaaggagagaacacccgcttgttacaccctgtgagcctgcat
gggatggatgacccggagagagaagtattagagtggaggtttgacagccgcctagcatttcatcacatggcccgagagctgcatccggactgt
actgggtctctctggttagaccagatctgagcctgggagctctctggctaactagggaacccactgcttaagcctcaataaagcttgccttgagtg
cttcaagtagtgtgtgcccgtctgttgtgtgactctggtaactagagatccctcagacccttttagtcagtgtggaaaatctctagcagcatgtgagc
aaaaggccagcaaaaggccaggaaccgtaaaaaggccgcgttgctggcgtttttccataggctccgcccccctgacgagcatcacaaaaatc
gacgctcaagtcagaggtggcgaaacccgacaggactataaagataccaggcgtttccccctggaagctccctcgtgcgctctcctgttccgac
cctgccgcttaccggatacctgtccgcctttctcccttcgggaagcgtggcgctttctcatagctcacgctgtaggtatctcagttcggtgtaggtc
gttcgctccaagctgggctgtgtgcacgaaccccccgttcagcccgaccgctgcgccttatccggtaactatcgtcttgagtccaacccggtaag
acacgacttatcgccactggcagcagccactggtaacaggattagcagagcgaggtatgtaggcggtgctacagagttcttgaagtggtggcct
aactacggctacactagaagaacagtatttggtatctgcgctctgctgaagccagttaccttcggaaaaagagttggtagctcttgatccggcaaa
caaaccaccgctggtagcggtggtttttttgtttgcaagcagcagattacgcgcagaaaaaaaggatctcaagaagatcctttgatcttttctacgg
ggtctgacgctcagtggaacgaaaactcacgttaagggattttggtcatgagattatcaaaaaggatcttcacctagatccttttaaattaaaaatga
agttttaaatcaatctaaagtatatatgagtaaacttggtctgacagttaccaatgcttaatcagtgaggcacctatctcagcgatctgtctatttcgttc
atccatagttgcctgactccccgtcgtgtagataactacgatacgggagggcttaccatctggccccagtgctgcaatgataccgcgagaccca
cgctcaccggctccagatttatcagcaataaaccagccagccggaagggccgagcgcagaagtggtcctgcaactttatccgcctccatccag
tctattaattgttgccgggaagctagagtaagtagttcgccagttaatagtttgcgcaacgttgttgccattgctacaggcatcgtggtgtcacgctc
gtcgtttggtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatccgtaagatg
cttttctgtgactggtgagtactcaaccaagtcattctgagaatagtgtatgcggcgaccgagttgctcttgcccggcgtcaatacgggataatacc
gcgccacatagcagaactttaaaagtgctcatcattggaaaacgttcttcggggcgaaaactctcaaggatcttaccgctgttgagatccagttcg
atgtaacccactcgtgcacccaactgatcttcagcatcttttactttcaccagcgtttctgggtgagcaaaaacaggaaggcaaaatgccgcaaaa
aagggaataagggcgacacggaaatgttgaatactcatactcttcctttttcaatattattgaagcatttatcagggttattgtctcatgagcggatac
atatttgaatgtatttagaaaaataaacaaataggggttccgcgcacatttccccgaaaagtgccacctgac.

MSCV-Cre was obtained from the laboratory of Luk Van Parijs. This vector to express Cre recombinase was a variant of pMIG in which the GFP reporter was replaced by the human surface molecule CD8 that has a deletion fo the cytoplasmic tail. Cre was cloned into this MICD8 vector by digestion with BglII and EcoRI and ligation.

To generate MCSV-Cre, pMIG (Grez et al., Proc. Natl. Acad. Sci. USA 87:9202-9206, 1990) was modified, as follows: the vector was digested with BglII and EcoRI, the Cre insert replaced ires-GFP, followed by insert fill in and ligation.

### Reagents

Anti p53 antibody was provided by Andrea Ventura. Doxorubicin and doxycycline were obtained from Sigma.

### Verification of Reporter Expression:

Lewis Lung carcinoma cells (LL2) were transducted with pFLIP and probed for Thy 1.1 expression by FACS analysis following puromycin selection. Selected cells were also probed for GFP expression, prior to and following infection with MCSV-Cre.

Infectious viral particles were produced through standard lab methods. 293FT cells were transiently transfected with retroviral gag/pol and VSVg envelopes plasmids along with the viral vector. The supernatants were harvested at 48 hours and used to infect target cells in the presence of 4 ug/ml polybrene.

### In Vitro Knockdown Studies

Primary mouse embryo fibroblasts (MEFs) were infected with MSCV retrovirus expressing pFLIP encoding RNAi against the tumor suppressor p53, or luciferase. Cells were selected with puromycin and infected with MSCV-Cre, treated for 5 hours with doxorubicin, and p53 expression was assayed by Western blotting. GAPDH served as a protein loading control.

### Example 1

### Construction of Stable, Cre-lox Based Knockdown Constructs

The pFLIP construct is schematically depicted in FIG. 1. The construct may be expressed by a constitutive, tissue-specific, or inducible promoter. The mRNA expresses puromycin resistance and the surface marker Thy1.1 or GFP. The puromycin-Thy1.1, or -GFP construct, respectively, is translated as a fusion protein but generates two distinct polypeptides by virtue of the 2A peptide at the C-terminus of the puromycin resistance or GFP cassette, which results in the translation of two distinct polypeptide species from a single cistron.

As depicted in FIG. 1, the green fluorescent protein (GFP) and a miR30 microRNA-based, RNAi construct are present in the anti-sense orientation in the 3′ untranslated region of the mRNA. Upon addition of Cre recombinase, the puro-Thy1.1 cassette is deleted and the GFP-microRNA construct is reversed to the sense orientation, allowing expression of GFP and RNAi.

In this embodiment, the vector expresses two markers a drug selection and a surface marker. When Cre is introduced, the markers are deleted and expression of GFP and RNAi is induced. GFP and the RNAi are in antisense orientation until Cre-recombinase is active, at which point there is a “flip” to the sense orientation.

### Example 2

### pFLIP Constructs are Stably Expressed Following Cre-Mediated Recombination

In order to determine whether the construct design in Example 1 results in conditional knockdown of specific gene expression, FACS analysis of marker expression was conducted on cells transduced with the pFLIP constructs described in an “uninduced” (no Cre recombinase supplied) and “induced” state (following Cre supply) (FIG. 2).

Lewis Lung carcinoma cell line (LL2) transduced with pFLIP-MSCV and selected with puromycin expressed the surface marker Thy1.1. (panel 2), in contrast to uninfected negative controls. These cells did not express GFP in the “uninduced” state, i.e. prior to exposure to MSCV-Cre (panel 3), while GFP expression was readily evident in cells infected with the Cre-expressing retrovirus (Panel 4). GFP expression was readily evident in MEF cells transduced with pFLIP-MSCV expressing the puromycin-GFP construct described in Example 1, when subjected to puromycin selection (FIG. 2, panel 5)

### Example 3

### Conditional Gene Knockdown With pFLIP Constructs

Primary mouse embryo fibroblasts (MEFs) expressing pFLIP encoding an RNAi against the tumor suppressor p53 conditionally knocked down p53 expression, when infected with MSCV retrovirus (FIG. 3). Cells treated with doxorubicin, which causes DNA damage and induces p53 expression, selected with puromycin demonstrated p53 expression, in “uninduced” conditions, however, when infected with a retrovirus expressing Cre recombinase, showed p53 knockdown, as seen by reduced p53 staining in Western Blots (lane 4).

Thus efficient gene knockdown was accomplished with the pFLIP constructs of this invention, specifically under conditions of Cre-mediated induction.

Knockdown was accomplished by recombinantly expressed miRNA. It is thought that while the miRNA sequence is in the antisense orientation, it can still fold to form a stem-loop structure, which is thought to be necessary for miRNA activity, thus background knockdown of gene expression might have been predicted with the constructs of this invention, yet surprisingly, this was not the case. The microRNA in antisense orientation was not processed to generate effective RNAi intermediates, nor did the antisense disrupt marker gene translation or virus production, indicating that the constructs provide for specific, controlled regulation of gene expression.

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

2.-22. (canceled)

23. A method of conditionally reducing expression of a coding sequence in a target cell, said method comprising contacting said target cell with a vector comprising:
i. a first pair of loxP sequences, inverted in orientation, with respect to each other;
ii. a first nucleic acid encoding a first selectable marker in sense orientation, wherein said nucleic acid is positioned between said first pair of loxP sequences;
iii. a second nucleic acid encoding a second selectable marker, fused in frame to an miRNA sequence of interest in antisense orientation, said second nucleic acid is positioned between said first pair of loxP sequences, and said second nucleic acid is 3′ with regard to said first nucleic acid;
iv. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

24.-45. (canceled)

46. A method of conditionally expressing a coding sequence in a target cell, the method comprising contacting said target cell with a vector comprising:
v. a first pair of loxP sequences, inverted in orientation, with respect to each other;
vi. a first nucleic acid encoding a first selectable marker in sense orientation, fused in frame to an miRNA sequence of interest, wherein said nucleic acid is positioned between said first pair of loxP sequences;
vii. a second nucleic acid encoding a second selectable marker, in antisense orientation, said second nucleic acid is positioned between said first pair of loxP sequences, and said second nucleic acid is 3′ with regard to said first nucleic acid;
viii. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.
wherein said cell expresses said miRNA agent, thereby reducing expression of said coding sequence and whereby, following Cre-mediated recombination in said target cell, said miRNA agent is no longer expressed, thereby being a method of conditionally expressing a coding sequence in a target cell.

47.-68. (canceled)

69. A vector comprising:
ix. a first pair of loxP sequences, inverted in orientation, with respect to each other;
x. a first nucleic acid encoding a first selectable marker in sense orientation, fused in frame to an miRNA sequence of interest, wherein said nucleic acid is positioned between said first pair of loxP sequences;
xi. a second nucleic acid encoding a second selectable marker, in antisense orientation, said second nucleic acid is positioned between said first pair of loxP sequences, and said second nucleic acid is 3′ with regard to said first nucleic acid; and
xii. a second pair of loxP sequences, inverted in orientation, with respect to each other, wherein said first loxP sequenced of said second pair is positioned between said first and said second nucleic acid, and said second loxP sequence in said second pair is positioned 3′ with respect to said first pair of loxP sequences, and said second pair of loxP sequences differs from that of said first pair of loxP sequences.

70.-86. (canceled)
